(19) United States
(12) Patent Application Publication
(10) Pub. No.: US 2025/0163387 A1
(43) Pub. Date: May 22, 2025

(54) HUMAN PITUITARY NEUROENDOCRINE TUMOR ORGANOIDS AND METHODS OF MAKING AND USING THE SAME

(71) Applicant: ARIZONA BOARD OF REGENTS OF THE UNIVERSITY OF ARIZONA, A BODY CORPORATE, Tucson, AZ (US)

(72) Inventors: Yana ZAVROS, Tucson, AZ (US); Andrew LITTLE, Tucson, AZ (US)

(21) Appl. No.: 18/839,222

(22) PCT Filed: Feb. 17, 2023
(86) PCT No.: PCT/US2023/062873

§ 371 Date: Aug. 16, 2024

Related U.S. Application Data
(60) Provisional application No. 63/268,251, filed on Feb. 18, 2022.

(51) Int. Cl. C12N 5/09; G01N 33/50

(52) U.S. Cl. CPC C12N 5/0693; G01N 33/5082; C12N 2503/00; C12N 2506/45; C12N 2510/00; G01N 2800/04

(57) ABSTRACT

A human patient-derived organoid derived from human pituitary neuroendocrine tumor and methods of preparing the same are disclosed. Drug screening platform to identify compounds or biologics for treatment of Cushing's disease (CD) patients is also provided. These studies make it possible for effective personalized medicine for these patients.

### CROSS-REFERENCE TO RELATED APPLICATION

This application claims priority to U.S. Provisional Application No. 63/268,251, filed on Feb. 18, 2022, the content of which is incorporated herein by reference in its entirety for all purposes.

### BACKGROUND

Cushing's disease (CD) is a serious endocrine disorder caused by dysregulated adrenocorticotropic hormone (ACTH)-secreting pituitary neuroendocrine tumor (PitNET) that stimulates the adrenal glands to overproduce cortisol [1-4]. The WHO renamed pituitary adenomas as PitNETs [5]. While PitNETs have been defined as benign, implying that these tumors cause a disease that is not life threatening or harmful to health, chronic exposure to excess cortisol has wide ranging and detrimental effects on health. Hypercortisolism causes increased stroke rates, diabetes, obesity, depression, anxiety and a threefold increase in the risk of death from cardiovascular disease and cancer [4,6] [7,8].

The first-line treatment for CD is pituitary surgery, which is followed by disease recurrence in 18% to 26% of patients during the 10-year follow-up period after surgery in the hands of an experienced surgeon [9-11]. Studies have demonstrated that surgical failures and late recurrences of CD are common, and despite multiple treatments, biochemical control is not achieved in approximately 50% of patients, suggesting that in routine clinical practice, initial and long-term disease remission is not achieved in a substantial number of CD patients [7,12]. Hence, medical therapy is often considered in the following situations: when surgery is contraindicated or fails to achieve remission, or when recurrence occurs after apparent surgical remission. While stereotactic radiosurgery treats incompletely resected or recurrent pituitary adenomas, the main drawbacks include the longer time to remission (12-60 months) and the risk of hypopituitarism [3,13,14]. There is an inverse relationship between disease duration and reversibility of complications associated with the disease thus emphasizing the importance of identifying an effective medical strategy to rapidly normalize cortisol production by targeting the pituitary adenoma [4,7,12]. Unfortunately, the lack of current standard of care treatments with low efficacy and tolerability makes CD a medical therapeutic challenge.

The overall goal of medical therapy for CD is to target the signaling mechanisms to lower cortisol levels in the body [15, 16]. The drugs offered for treatment of CD vary in the mechanism of action, safety, tolerability, route of administration, and drug-drug interactions [15, 16]. The absence of preclinical models that replicate the adenoma tissue has prevented us from acquiring the knowledge to advance clinical care by implementing therapies specifically targeting the adenoma with a higher efficacy and tolerability for CD patients. In this instance, organoids can replicate much of the complexity of an organ. For purpose of this disclosure, an “organoid” is a 3-dimensional cell structure, grown from primary cells of dissociated pituitary tumors, that self-renew, proliferate and differentiate in 3 dimensions, eventually replicating key biological properties of the organ/tissue. While pituitary cell lines predominantly represent hormonal lineages, these cultures do not reproduce the primary pituitary tissue because of the tumor transformation and non-physiological 2D culture conditions [19-21]. Pituitary tissue-derived organoids have only been generated from mouse models [22,23]. While several human and rat pituitary spheroid/aggregate/tumoroid models have been reported, these cultures consist of poorly differentiated cells with high replicative potential that can affect drug response and produce data that poorly translate to the clinic [24,25]. This disclosure provides a human organoid model derived from human PitNETs that replicated much of the cellular complexity and functionality of the patient's tumor.

### SUMMARY

The present disclosure provides a human organoid model derived from human PitNETs that replicated much of the cellular complexity and functionality of the patient's tumor and methods of generating these organoids.

In one embodiment, an organoid derived from a pituitary neuroendocrine tumor (PitNET) of a human subject is disclosed, wherein the organoid comprises a plurality of cells that is well differentiated, and wherein the organoid possesses structure and function similar to those of the PitNET.

In one aspect, the PitNET is selected from the group consisting of a corticotroph adenoma, a lactotroph adenoma, a gonadotroph adenoma, and a somatotroph adenoma. In another aspect, the PitNET secrets adrenocorticotropic hormone (ACTH).

In another embodiment, at least some cells of the organoid retain genetic alteration(s) of the human subject's primary tissue.

In some other embodiments, at least some cells of the organoid carry an M415V and/or an M415 mutation in the USP48 gene.

In some other embodiments, the PitNET is from a subject that carries a mutant allele selected from the group consisting of CDH23 and MEN1.

In some other embodiments, a method of treating Cushing's Disease (CD) caused by a pituitary neuroendocrine tumor (PitNET) in a subject is disclosed, which includes the following steps:
a) extracting a plurality of cells from the PitNET of the subject,
b) culturing the plurality of cells to form an organoid,
c) contacting the organoid with a compound selected from a plurality of compounds,
d) determining if the compound increases apoptosis or reduces hormonal secretion of the organoid after being contacted with the compound, and
e) selecting the compound that increases apoptosis or reduces hormonal secretion of the organoid as a candidate compound,
f) administering the candidate compound to the subject.

In one aspect, steps (c) and (d) above are repeated until all compounds from the plurality of compounds have gone through steps (c) and (d).

In another aspect, the candidate compound binds to human glucocorticoid receptor (GR) but does not bind to other non-GR hormone receptors.

In another aspect, the candidate compound is administered together with a pharmaceutically acceptable carrier.

In some other embodiments, an organoid is disclosed comprising a plurality of cells derived from a human induced pluripotent stem cells (iPSC) lines, wherein somatic mutations M415V and/or M415I in the gene USP48 have been introduced into certain cells of the organoid. In one aspect, the somatic mutations M415V and/or M415I are introduced by CRISPR.

In some other embodiments, a method of using the disclosed organoid to screen for bioactive compounds is disclosed. The method may include at least these steps: a) contacting any of the organoid described herein with one or more compounds, b) assessing apoptosis or hormonal secretion profile of the organoid after the contacting step (a), and c) identifying a candidate compound that induces apoptosis or reduces ACTH secretion.

### BRIEF DESCRIPTION OF THE FIGURES

The following figures form part of the present specification and are included to further illustrate aspects of the present invention.

FIG. 1 shows Morphology and function of corticotroph hPITOs. (a-g) Brightfield images, immunofluorescence staining using antibodies specific for CAM5.2 (red), ACTH (green), and EdU (magenta, inset) of organ-oid cultures generated from patients with Cushing's disease (hPITOs 1, 7, 10, 33, 35) or nonfunctional corticotroph adenomas (hPITO8, 12). Quantification of % EdU positive cells/total cell number is shown and compared to the Ki67 score given in the pathology report (Supplemental Table 3). An ELISA was performed using conditioned media collected from (h) corticotroph hPITO cultures and (i) lactotroph, somatotroph and gonadotroph hPITO cultures for the measurement of ACTH secretion (pg/ml).

FIG. 2 shows Cell heterogeneity of corticotroph hPITOs. (a) viSNE maps define spatially distinct cell populations using pituitary specific cell lineage, stem cell and transcription factor markers. Cell populations were quantified in organoids generated from CD patients with corticotroph adenomas (sparsely granulated and Crooke's cell adenoma) or patients with nonfunctional corticotroph adenomas. (b) Quantification of the abundance of cells expressing pituitary specific markers as percent total. viSNE maps define spatially distinct cell populations in organoid cultures generated from CD patient with (c) corticotroph adenoma (hPITO37, Crooke's cell adenoma) and adjacent normal tissue (hPITO37N) or a (d) sparsely granulated corticotroph adenomas (hPITO38) and adjacent nor-mal tissue (hPITO38N).

FIG. 3 shows Drug screen using hPITOs generated from CD patients. (a) High-throughput drug screening of hPITOs reveals sensitivities to a range of therapeutic agents. Cell viability with high values (indicating resistance) are depicted in red, and low values (indicating sensitivity) are in blue in the clustered heatmap. (b, c) Clusters showing response to therapeutic agents with most variance across the organoids. (d) Network of drugs from the clusters b and c and their gene targets showing their participation in signaling pathways and cellular processes.

FIG. 4 shows Drug dose responses by hPITOs generated from CD patients. Dose responses to mifepristone, GANT61, cabergoline and osilodro-stat (a, e) hPITO28, (b, f) hPITO33, (c, g) hPITO34, and (d, h) hPITO35. Dose responses to cabergoline, ketoconazole, roscovitine, GANT61, pasireotide, mifepristone, etomidate, mitotane, metyrapone and osilodrostat in (i) hPITO37 and (j) organoids generated from adjacent normal pituitary tissue (hPITO37N), and (k) hPITO38 and (l) hPITO38N, and (m) hPITO39. (n) IC50 and integrated area under the curve in response to mifepristone, ketoconazole and pasireotide using hPITO39 cultures. Nuclear morphometric analysis of hPITO39 cultures in response to (o, p) vehicle, (q, r) mifepristone, (s, t) pasireotide, and (u, v) ketoconazole. Morphometric classification of NII was based on the normal (N), small(S), small regular (SR), short irregular (SI), large regular (LR), large irregular (LI) and irregular (I) nuclear morphology. Representative Hoechst staining of organoids in response to drug treatments for the calculation of nuclear irregularity index (NII) are shown in the insets in p, r, t and v.

FIG. 5 shows SSTR1-5 expression in hPITOs and patient's PitNET tissue. (a) Dose response of hPITO28, 31, 33, 34, and 35 lines to pasireotide. (b) Differential expression of SSTR subtypes 1-5 (SSTR1, SSTR2, SSTR3, SSTR4, SSTR5) in hPITO28, hPITO31, hPITO33, hPITO34, and hPITO35. Immunohistochemistry of (c, e) SSTR2 and (d, f) SSTR5 expression in patient PitNET tissue (Pt28 and Pt34) from which hPITO28 and 34 were generated.

FIG. 6 shows Genomic landscape of hPITOs recapitulates genetic alterations commonly found PitNETs. Overview of single nucleotide variation events detected in hPITOs in genes commonly altered in PitNETs. The mutation frequency across the organoid population is depicted on the right. Color coding of the figure shows organoid lines are derived from the same patient tumor tissue. ORG: organoid line, TIS: matched patient's PitNET tissue.

FIG. 7 shows Single cell analysis of iPSCctrl and iPSCCDH23 cultures 15 and 30 days post-directed differentiation. (a) UMAP plots showing identified cell clusters 0-16 in iPSCctrl and iPSCCDH23 cultures 15 days post-directed differentiation. (b) Violin plots of representative identified markers of the corticotroph cell lineage where 2 subpopulations were observed among iPSCctrl and iPSCCDH23 cultures. Arrows highlight clusters 1, 5 and 7. (c) Violin plots showing expression of genes representative of stem cells, Wnt, NOTCH, Hh and SST signaling, anterior pituitary (corticotroph) cell lineage and cell cycle in clusters 1, 5 and 7 of iPSCCDH23 cultures. plot width: cell number, plot height: gene expression. (d) viSNE maps showing concatenated flow cytometry standard files for both samples and iPSCctrl and iPSCCDH23 organoids 30 days post-directed differentiation. (e) Overlay of manually gated cell populations on to viSNE plots. (f) Fluorescent intensity of Ki67 of viSNE maps for both samples and iPSCctrl and iPSCCDH23 organoids. iPSCctrl=22518 events; iPSCCDH23=17542 events.

FIG. 8 illustrates Morphology and proliferation of lactotroph, somatotroph and gonadotroph hPITOs. (a) Bright-field images, and (b) immunofluorescence staining using antibodies specific for EdU (magenta) of organoid cultures generated from patients diagnosed with lactotroph, somatotroph and gonadotroph adenomas. (c, d, e) Quantification of % EdU positive cells/total cell number is shown and compared to the Ki67 score given in the pathology report.

FIG. 9 shows KaryoStat™ analysis of (a) iPSCCDH23 and (b) iPSCMEN1 lines. No chromosomal aberrations were found in either line when comparing against the reference dataset.

FIG. 10 shows a differentiation schedule for the generation of pituitary or ganoids derived from iPSCs. Pituitary organoids were generated based on the outlined schedule using iPSCs generated from PBMCs collected from CD patients, or a healthy individual. Bright field images demonstrating morphological variation is observed between iPSCctrl and iP-SCCDH23 lines and organoids.

FIG. 11 shows Familial germline mutations found in CD patients for the generation of iPSC lines. (A) Amino acid and nucleotide sequence for the germline MEN1 L444P mutation, in exon 9. Also shown are the gene coordinates in the whole MEN1 gene for the subsequence present. (B) Restriction Fragment Length Polymorphism (RFLP) validation to con-firm that iPSCMEN1 harbored the correct mutation compared to control. Gel images compared undigested (U) DNA to samples digested with the restriction enzyme EcoRII (E). The MEN1 mutation introduced 2 new EcoRII sites, which is present in the iPSCMEN1 gel. iPSCCtrl displayed no separation of bands in digested versus undigested samples. (C) Amino acid and nucleotide sequence for two germline CDH23 mutations, G490A (exon 15) and A1222T (exon 31) observed in iPSCCDH23. Also shown are the gene coordinates in the entire CDH23 gene for the subsections present.

FIG. 12 shows Expression pattern of major hormone-producing cell lineages in iPSCs differentiated topituitary organoids. Expression of PIT-1 (green), ACTH (green), GH (red), FSH (red), LH (green), PRL (red) and synaptophysin (synapto, green) with co-stain Hoechst (nuclei, blue) was measured by immunofluorescence using chamber slides collected at day 15 (D15) of the differentiation schedule of control iPSCs (iPSCctrl, a, b) and iPSCs expressing the MEN1 (IP-SCMEN1 c, d) and CDH23 (iPSCCDH23, e, f) mutations. Red arrows highlight the increased expression of ACTH and synaptophysin with the concomitant loss of PIT1, GH, FSH, LH and PRL in iPCSs expressing mutated MEN1. Inset in c is a higher magnification of synaptophysin.

FIG. 13 shows Single cell analysis of iPSCctrl and iPSCCDH23 cultures 15 days post-directed differentiation. (a) An ELISA was performed using conditioned media collected during the differentiation schedule from iPSCctrl and iPSCCDH23 cultures for the measurement of ACTH secretion (pg/mL). * P<0.05 compared to iPSCctrl organoid line, n=3 individual experimental replicates.

FIG. 14 shows Supplemental Table S1. Pituitary Organoid Growth Media.

FIG. 15 shows Supplemental Table S2. Induced Pluripotent Stem Cell Generated Pituitary Organoid Growth Media.

FIG. 16 shows Supplemental Table S3. Clinical Characteristics of Pituitary Adenoma Samples Used for the Generation of Organoids.

FIG. 17 shows Expression pattern of pituitary hormone-producing cell lineages in iPSCs differentiated to pituitary organoids. Expression of PIT-1 (green), ACTH (green), GH (red), FSH (red), LH (green), PRL (red) and synaptophysin (synapto, green) with co-stain Hoechst (nuclei, blue) was measured by immunofluorescence using chamber slides collected at day 15 (D15) of the differentiation schedule of (A) control iPSCs (iPSCctrl) and iPSCs expressing (B) USP48M415V (iPSC^{USP48MV}), (C) USP48M415I (iPSC^{USP48MI}) and (D) USP8 (iPSC^{USP8}) mutations. Red arrows highlight the increased expression of ACTH and synaptophysin with the concomitant loss of PIT1, GH, FSH, LH and PRL in iPCSs expressing somatic mutations USP48 and USP8. Quantification of the percentage of positive cells is shown in the dot plots for each iPSC line. * P<0.05 compared to the PIT1, GH, FSH, LH and PRL cell lineages for each line. (E) Expression of PIT-1 (green), ACTH (green), GH (red), FSH (red), LH (green), PRL (red) and synaptophysin (synapto, green) with co-stain Hoechst (nuclei, blue) was measured by immunofluorescence using positive (pituitary tissue) and negative (gastric tissue) controls. Quantification for positive and negative controls is shown to the right.

FIG. 18 shows Expression of cell linages and transcription factors in iPSC generated pituitary tumor organoids. (A) Immunohistochemistry using FFPE sections prepared from iPSCctrl, iPSC^{USP48MV}, iPSC^{USP48MI}, and iPSC^{USP8} organoids stained with antibodies specific for CAM5.2, T-Pit, Synaptophysin and ACTH. High magnification images are shown in insets. (B) Differential expression of transcription factors TPit, PIT1 and SF1 and POMC were measured by qRT-PCR. (C) Plot comparing the ACTH secretion (pg/ml), measured by an ELISA using conditioned media of iPSCctrl, iPSC^{USP48MV}, iPSC^{USP48MI}, and iPSC^{USP8} throughout the directed differentiation schedule. (D) viSNE maps showing concatenated flow cytometry standard files for iPSCctrl, iPSC^{USP48MV}, iPSC^{USP48MI}, and iPSC^{USP8} organoids 30 days post-directed differentiation. Maps define spatially distinct cell populations using pituitary specific cell lineage, stem cell and transcription factor markers between iPSCctrl organoids and mutant lines. (E) viSNE heatmaps showing the number of cells positive for proliferation marker Ki67. (F) Violin plots comparing the TPit lineage expression, through proliferation marker Ki67 between control and mutated iPSC lines. *p<0.05 compared to iPSCctrl organoids, n=3 individual experimental replicates.

FIG. 19 shows Differential expression of SSTR2 and SSTR5 in iPSC generated pituitary tumor organoids in response to Mifepristone and Relacorilant. Differential expression of (A) SSTR2 and (B) SSTR5 in iPSC^{ctrl}, iPSC^{USP48MI}, iPSC^{USP48MV} and iPSCUSP8 organoids in response to vehicle (Veh), Mifepristone (Mife, 500 nM) or relacorilant (Rela, 500 nM). * p<0.05 compared to vehicle-treated organoids, #p<0.05 compared to Mifepristone treated organoids, n=3 experimental replicates/organoid line. (C) Differential expression of SSTR2 and SSTR5 in iPSC^{ctrl} organoids treated with vehicle (Veh), Mifepristone (Mife), GANT61 (GANT, 5 uM), Mife+GANT, ketoconazole (Keto, 10 uM), Mife+Keto, or dexamethasone (Dexa, 100 nM). * p<0.05 compared to iPSCctrl organoids treated with Veh, n=3 experimental replicates/organoid line. (D) Differential expression of SSTR2 and SSTR5 in iPSCctrl organoids treated with vehicle (Veh), relacorilant (Rela), GANT61 (GANT), Rela+GANT, ketoconazole (Keto), Rela+Keto, or dexamethasone (Dexa). * p<0.05 compared to iPSCctrl organoids treated with Veh, n=3 experimental replicates/organoid line. (E) Mutations in USP48 and USP8 in PitNETs are believed to enhance corticotropin releasing hormone (CRH)-induced production coherent with the Hh signaling pathway. Hh ligand, Sonic Hedgehog (Shh), binds to Patched (Ptch1) that relieves suppression of Smoothened (SMO) and subsequently Gli1 activation. Crosstalk between Shh and CRH at the Gli1 level stimulates POMC transcription and ACTH secretion. (F) Differential expression of POMC in iPSC^{ctrl}, iPSC^{ctrl}, iPSC^{USP48MI}, iPSC^{USP48MV} and iPSC^{USP8} organoids. * p<0.05 compared to iPSCctrl organoids, n=3 experimental replicates/organoid line. (G) Representative western blots of the expression of Gli1 relative to GAPDH in iPSCctrl, iPSC^{USP48MI}, iPSC^{USP48MV} and iPSC^{USP8} organoids. (H) Quantification of western blots shown in G. * p<0.05 compared to iPSCctrl organoids, n=3 experimental replicates/organoid line. (I) Mutations in USP8 and USP48 detected in hPITO cultures (hPITO1 and 7) were also expressed in the patient's matched PitNET tissue. (J) Human PITO cultures expressing USP8 and USP48 mutations were used for gene ChIP analysis after treatment with vehicle or GANT61. * p<0.05 compared to iPSCctrl organoids, n=4 experimental replicates/organoid line.

FIG. 20 shows Changes in pituitary tumor cell proliferation, viability and ACTH secretion in iPSC^{ctrl} organoids in response to Mifepristone and relacorilant. (A) Immunofluorescence images of EdU expression in iPSC^{ctrl} organoids in response to vehicle (Veh), Mifepristone (Mife, 500 nM), Pasireotide (Pas, 100 nM), Octreotide (Oct, 100 nM), relacorilant (Rela, 500 nM), Mife+Pas, Mife+October, Rela+Pas, Rela+October, dexamethasone (Dexa, 100 nM). (B) Quantification of EdU positive cells of iPSC^{ctrl} and mutant organoids. * p<0.05 compared to iPSC^{ctrl} organoids, n=4 individual organoids quantified per culture. (C) Representative Hoechst staining of iPSC^{ctrl} organoids in response to experimental treatments for the calculation of nuclear irregularity index (NII). Nuclear morphometric analysis of iPSC^{ctrl} organoids in response to experimental treatments with quantification shown for (D) Veh, (E) Mife, or (F) Rela treatments. Morphometric classification of NII was based on the normal (N), small(S), small regular (SR), short irregular (SI), large regular (LR), large irregular (LI) and irregular (I) nuclear morphology. (G) An ELISA was performed using conditioned media collected from iPSC^{ctrl} cultures in response to treatments for the measurement of ACTH secretion (pg/ml). * p<0.05 compared to Veh treatment, #p<0.05 compared to Mife or Rela alone, n=4 individual organoids quantified per culture.

FIG. 21 shows Changes in pituitary tumor cell proliferation, viability and ACTH secretion in iPSC^{USP48MV} organoids in response to Mifepristone and relacorilant. (A) Immunofluorescence images of EdU expression in iPSC^{USP48MV} organoids in response to vehicle (Veh), Mifepristone (Mife, 500 nM), Pasireotide (Pas, 100 nM), Octreotide (Oct, 100 nM), relacorilant (Rela, 500 nM), Mife+Pas, Mife+October, Rela+Pas, Rela+October, dexamethasone (Dexa, 100 nM). (B) Quantification of EdU positive cells of iPSC^{USP48MV} and mutant organoids. * p<0.05 compared to iPSC^{USP48MV} organoids, n=4 individual organoids quantified per culture. (C) Representative Hoechst staining of iPSC^{ctrl} organoids in response to experimental treatments for the calculation of nuclear irregularity index (NII). Nuclear morphometric analysis of iPSC^{USP48MV} organoids in response to experimental treatments with quantification shown for (D) Veh, (E) Mife, or (F) Rela treatments. Morphometric classification of NII was based on the normal (N), small(S), small regular (SR), small irregular (SI), large regular (LR), large irregular (LI) and irregular (I) nuclear morphology. (G) An ELISA was performed using conditioned media collected from iPSC^{USP48MV} cultures in response to treatments for the measurement of ACTH secretion (pg/ml). * p<0.05 compared to Veh treatment, #p<0.05 compared to Mife or Rela alone, n=4 individual organoids quantified per culture.

FIG. 22 shows Changes in SSTR2, SSTR5 and ACTH expression, and pituitary tumor cell death in iPSC^{USP48MV} organoids in response to Mifepristone and relacorilant. Fluorescent intensity of (A) SSTR2, (B) SSTR5, (C) ACTH, and (D) zombie of viSNE heatmaps for iPSC^{USP48MV} organoids in response to vehicle (Veh), Pasireotide (Pas, 100 nM), relacorilant (Rela, 500 nM), Rela+Pas, Mifepristone (Mife, 500 nM), and Mife+Pas. iPSC^{USP48}=15000 events. Quantification of percentage of SSTR2 (E), SSTR5 (F), ACTH (G), or Zombie (H) positive cells within iPSC^{USP48MV} organoid cultures in response to experimental treatments. * p<0.05 compared to Veh.

FIG. 23 shows Changes in SSTR2, SSTR5 and ACTH expression, and pituitary tumor cell death in hPITOs in response to Mifepristone and relacorilant. (A) Brightfield images of organoid cultures generated from patients with CD (hPITOs 37, 38, 39, 40). (B) Immunofluorescence staining using antibodies specific for CAM5.2 (red) and ACTH (green) of hPITO37. Immunohistochemical staining using antibodies specific for ACTH and CAM5.2 of FFPE sections prepared from embedded organoid line (left panels, hPITO37), and patient's matched PitNET tissue (Pt37, right panels). (C) viSNE maps showing concatenated flow cytometry standard files for hPITO37 line defining the spatially distinct cell populations using pituitary specific cell lineages, stem cell and transcription factor markers. (D) Differential expression of SSTR2 and SSTR5 measured in hPITO36, 37, 38, 39 and 40 lines. Fluorescent intensity of (E, F) SSTR2, (G, H) SSTR5, (I, J) ACTH, and (K, L) zombie of viSNE heatmaps for hPITO37 organoids in response to vehicle (Veh), Pasireotide (Pas, 837.5 nM), relacorilant (Rela, 382 nM), Rela+Pas, Mifepristone (Mife, 2.85 nM), and Mife+Pas. hPITO=5000 events. Quantification of percentage of SSTR2, SSTR5, ACTH, or Zombie positive cells within hPITO cultures in response to experimental treatments is shown in F, H, J and L. * p<0.05 compared to Veh.

FIG. 24 shows Analysis of Area-Under-Curve (AUC) for PitNET organoids treated with Pasireotide, Mifepristone or relacorilant. Dose response curves using PitNET organoid lines (hPITO36, 37, 38, 39 and 40) treated with (A) Pasireotide, (D) Mifepristone or (G) relacorilant. Dose response curves using PitNET organoid lines generated from adjacent normal tissue (hPITO37N, 38N) treated with (B) Pasireotide, (E) Mifepristone or (H) relacorilant. (C, F, I) Calculated AUC and IC50 values. n=3 experimental replicates performed per organoid line.

FIG. 25 shows Table 1 which summarizes the neuropathology reports and clinical diagnosis from cases used to generate hPITOs 37, 38, 39, 40 reported in the current study.

### DETAILED DESCRIPTION

Cushing's disease (CD) is a serious endocrine disorder caused by dysregulated adrenocorticotropic hormone (ACTH)-secreting pituitary neuroendocrine tumor (PitNET) that stimulates the adrenal glands to overproduce cortisol. It is disclosed here development of organoids generated from human pituitary tumors or induced pluripotent stem cells as an essential approach to identify targeted therapy for CD patients.

The absence of preclinical models that replicate the pituitary neuroendocrine tumor (PitNETs) microenvironment has prevented us from acquiring the knowledge to identify therapies that are targeted to the tumor with a higher efficacy and tolerability for patients. Our studies demonstrate the development of organoids generated from human pituitary tumors (hPITOs). Human PITOs can potentially be used to screen for the sensitivity and efficacy of responses to targeted therapies for CD patients that either fail to achieve remission or exhibit recurrence of disease after surgery. In addition, induced pluripotent stem cells (iPSCs) generated from CD patients expressing germline mutations CDH23 (iPSCCDH23) reveal the disease pathogenesis under directed differentiation. Many early in vitro experiments have used pituitary cell lines, spheroids, aggregates and/or tumoroids that do not replicate the primary PitNET microenvironment partly due unphysiological 2D culture conditions [19-21], and lack a multicellular identity [39,40]. The development of pituitary tissue generated organoids, are limited to the use of transgenic mouse models as the source [22,23,41]. The recent organoid cultures reported by Nys et al. have been generated from single stem cells isolated from PitNET tissue as claim of true organoids due to the clonality. However, the multicellular complexity was not validated by the protein expression or hormone secretion from pituitary cell lineages in these cultures [42]. According to the National Cancer Institute (NCI, NIH), an ‘organoid’ is defined as “A tiny, 3-dimensional mass of tissue that is made by growing stem cells (cells from which other types of cells develop) in the laboratory.” [43]. The hPITOs reported here begin from single and/or 3-4 cell clusters dissociated from the PitNET tissue that harbor the stem cells. The process involves ‘budding’, and lumen formation as organoids grow and differentiate. See Chakrabarti, et al., Cells 2022, 11, 3344. Differentiation and function were documented by comprehensive spectral flow cytometry, ELISA and response to standard of care drugs. Growth of PitNET organoids reported in the current study are consistent with gastrointestinal tissue derived cultures that begin from cell clusters, crypts, or glands [27,44,45].

In one embodiment, it is reported here a pituitary tumor organoid culture with a multicellular identity consisting of differentiated cell lineages, stem/progenitor cells, and immune and stromal cell compartments that replicates much of the patient's own adenoma pathology, functionality, and complexity. It is also demonstrated that iPSCs, derived from the blood of CD patients can be directly differentiated into pituitary organoids that resemble similar characteristics to the adenoma tissues. Many investigators have proposed using organoids in personalized medicine but have focused these efforts on targeted treatment of cancers [27,46-48]. The findings reported in these studies are the first to implement this approach for the potential treatment of pituitary adenomas. Collectively, we have developed a human relevant in vitro approach to potentially advance our knowledge and approach for studies in the field of pituitary tumor research. Both the hPITOs and iPSCCDH23 may be implemented in studies that strive to 1) define the molecular and cellular events that are crucial for the development of pituitary adenomas leading to CD, and 2) accelerate the identification of effective targeted therapies for patients with CD.

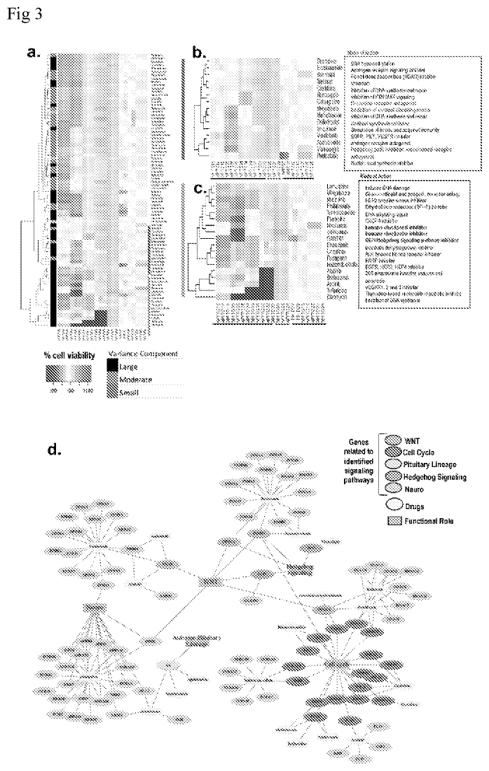

While published studies have advanced our understanding of the molecular mechanisms of the pathogenesis of corticotroph adenomas and elucidate candidate therapeutic targets for CD, these reports fall short of directly informing clinical decision for the patient's treatment. Using organoids to screen potential drugs and compounds can potentially improve therapeutic accuracy. FIG. 3 demonstrated a variation in drug responsiveness amongst the organoid lines generated from individual patients. Importantly, there was further divergence in drug responsiveness amongst the individual organoid lines within each pathologically defined corticotroph subtype. For example, hPITOs generated from patients with sparsely granulated corticotroph adenomas (hPITOs 10, 25, 34, 35) and Crooke's cell adenomas (hPITOs 7, 33) showed variable responses regardless of similar pathologically defined subtype. In addition, the response of the adenoma cells within the organoids to the standard of care drug that targets the pituitary directly in the body including mifepristone and cabergoline, is only an unacceptable 50% in hPITO34 and hPITO35 and almost 0% in other line including hPITO7, 10 and 25. These data clearly demonstrate that the inherent patient difference to drug response that is often observed among CD patients is reflected in the organoid culture.

In one embodiment, this culture system represents an approach that will provide functional data that would reveal actionable treatment options for each patient. Patient-derived organoids from several tumors has served as a platform for testing the efficacy of anticancer drugs and predict responses to targeted therapies in individual patients [27,46,48-50]. This study is the first report of the use of hPITOs for drug screening. Connecting genetic and drug sensitivity data will further categorize corticotroph subtypes associated with CD. WES analysis of each hPITO line was compared to the results for the corresponding primary adenoma tissues. We showed the concordance rate of exonic variants between the primary tumor tissues obtained from CD patients and the corresponding organoid line. On average, approximately 80% of the variants observed in the CD patient's adenoma tissue were retained in the corresponding hPITOs.

Pituitary organoids were also developed from iPSCs generated from PBMCs of a CD patient expressing a germline genetic alteration in cadherin-related 23 CDH23 (iPSC^{CDH23}), a CD patient expressing an MEN1 mutation (iPSCCDH23) and a healthy individual (iPSC^{ctrl}). Foundational studies performed by investigators at the genome level have revealed significant knowledge regarding the pathophysiology of CD [36,37,52,53]. In some instances, CD is a manifestation of genetic mutation syndromes that include multiple endocrine neoplasia type 1 (MEN1), familial isolated pituitary adenoma (FIPA) and Carney complex [54,55]. CDH23 syndrome is clinically associated with the development of Usher syndrome, deafness, and vestibular dysfunction [56]. Several mutations in CDH23 are associated with inherited hearing loss and blindness [57]. However, none of the variants found in this study were linked to any symptoms of deafness or blindness. A possible explanation is that deafness related CDH23 mutations are caused by either homozygous or compound heterozygous mutations [57]. In a study that linked mutations in CDH23 with familial and sporadic pituitary adenomas suggested that these genetic alterations could play important roles in the pathogenesis of CD [38]. Genomic screening in a total of 12 families with familial, and 125 individuals with sporadic pituitary adenomas, and 260 control individuals showed that 33% of the families with familial pituitary adenomas and 12% of individuals with sporadic pituitary adenomas expressed functional or pathogenic CDH23 variants [38]. Consistent with the expected pathology and function of pituitary adenoma from a patient with CD, iPSC^{CDH23} organoids exhibited hypersecretion of ACTH, and expression of transcription factors and cell markers reported in the pathology report for corticotroph adenomas. Collectively, these findings warrant the further investigation to determine if carriers of CDH23 mutations are at a high risk of developing CD and/or hearing loss. Specifically, clinical investigation is required to determine whether pituitary MRI scans should be adopted in the screening of CDH23-related diseases, including Usher syndrome and age-related hearing loss.

Pituitary organoids generated from iPSCs of CD patients revealed the existence of cell populations that potentially contribute to the support of adenoma growth and progression, and an expansion of stem and progenitor cells that may be the targets for tumor recurrence. Organoids derived from both pituitary adenomas and iPSCs exhibited increased expression of stem cell and progenitor markers at both the protein and transcriptomic levels. Unique clusters that were proliferative in the iPSC^{CDH23} organoids expressed a hybrid pituitary cell population that was in an epithelial/mesenchymal state (CK20+/VIM+/CXCR4+/Ki67+). In support of our findings, a similar report of a hybrid epithelial/mesenchymal pituitary cell has been made as part of the normal developmental stages of the human fetal pituitary [58]. Previous studies have suggested that pituitary stem cells undergo an EMT-like process during cell migration and differentiation [59-61]. Consistent with our findings are extensive studies using single cells isolated from human pituitary adenomas to show increased expression of stem cells markers SOX2 and CXCR4 [22,23,41,62,63]. Within the clusters identified in the iPSCCDH23 culture were cell populations expressing stem cell markers including SOX2, NESTIN, CXCR4, KLF4, and CD34. The same iPSC^{CDH23} cell clusters 4, 8, 9 and 11 co-expressed upregulated genes of NOTCH, Hedgehog, WNT and TGFb signaling that are pivotal in not only pituitary tumorigenesis and pituitary embryonic development, but also ‘tumor stemness’ [22,23,41,62-64]. It is also noted that clusters of cell populations 5 and 14 unique within the iPSCCDH23 cultures expressed upregulated genes indicative of high proliferation. We observed upregulated expression of the E2F family of transcription factors (E2Fs) E2F1 and E2F7. These findings are of significance given that there is evidence to show that upregulation of E2Fs is fundamental for tumorigenesis, metastasis, drug resistance and recurrence [65]. Within the pituitary adenoma microenvironment, whether these stem cells directly differentiate into pituitary tumors, or support the growth of the adenoma is largely unknown. In addition, whether pituitary stem cell populations become activated in response to injury is also understudied. Although the initiation of tumorigenesis of pituitary stem cells has been identified using a mouse model of implantation of cell within the right forebrain [66], the identification of pituitary tumor-initiating stem cells using the gold standard in vivo orthotopic transplantation model is impossible. Pituitary adenomas harboring the tumor stem cells may require engraftment within the environment from which the cells are derived to enable growth and differentiation of the tumor. However, it is technically impossible to implant cells orthotopically in the pituitary. The pituitary adenoma organoid cultures presented in these studies may offer an approach by which isolation, identification and characterization of this stem cell population is possible. Therefore, we would gain knowledge on the mechanisms of pituitary tumor pathogenesis and reveal potential novel targets for therapeutic interventions by using the iPSC generated pituitary organoid culture.

PitNETs associated with the development of CD cause serious morbidity due to chronic cortisol exposure that dysregulates almost every organ system in the body. Overall, existing medical therapy remains suboptimal with negative impact on health and quality of life, including considerable risk of therapy resistance and tumor recurrence. To date, little is known on the pathogenesis of pituitary tumors. Here, we present a human organoid-based approach that will allow us to acquire knowledge of the mechanisms underlying pituitary tumorigenesis. Such an approach is essential to identify targeted treatments and improve clinical management of patients with CD.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

The terms “polypeptide,” “peptide” and “protein” may be used interchangeably in this disclosure. The terms “oligonucleotide,” and “polynucleotide” may also be used interchangeably in this disclosure.

The term “organoid” refers to a miniaturized version of an organ produced in vitro in two or three dimensions by a group of cells that shows realistic micro-anatomy similar to an organ.

The instant disclosure is further described by the following items:

Item 1. An organoid derived from a pituitary neuroendocrine tumor (PitNET) of a human subject, wherein the organoid comprises a plurality of cells that is well differentiated, and wherein the organoid possesses structure and function similar to those of the PitNET.

Item 2. The organoid of Item 1, wherein the PitNET is selected from the group consisting of a corticotroph adenoma, a lactotroph adenoma, a gonadotroph adenoma, and a somatotroph adenoma.

Item 3. The organoid of Item 1, wherein the PitNET is a corticotroph adenoma.

Item 4. The organoid of any of preceding items, wherein the PitNET secrets adrenocorticotropic hormone (ACTH).

Item 5. The organoid of any of preceding items, wherein at least some cells of the organoid retain genetic alteration(s) of the human subject's primary tissue.

Item 6. The organoid of any of preceding items, wherein at least some cells of the organoid carry an M415V and/or an M415I mutation in the USP48 gene.

Item 7. The organoid of any of preceding items, wherein the PitNET is from a subject that carries a mutant allele selected from the group consisting of CDH23 and MEN1.

Item 8. A method of treating Cushing's Disease (CD) caused by a pituitary neuroendocrine tumor (PitNET) in a subject, comprising
a) extracting a plurality of cells from the PitNET of the subject,
b) culturing the plurality of cells to form an organoid,
c) contacting the organoid with a compound selected from a plurality of compounds,
d) determining if the compound increases apoptosis or reduces hormonal secretion of the organoid after being contacted with the compound, and
e) selecting the compound that increases apoptosis or reduces hormonal secretion of the organoid as a candidate compound,
f) administering the candidate compound to the subject.

Item 9. The method of Item 8, wherein steps (c) and (d) are repeated until all compounds from the plurality of compounds have gone through steps (c) and (d).

Item 10. The method of any of Items 8-9, wherein the candidate compound binds to human glucocorticoid receptor (GR) but does not bind to other non-GR hormone receptors.

Item 11. The method of any of Items 8-10, wherein the candidate compound is administered together with a pharmaceutically acceptable carrier.

Item 12. An organoid comprising a plurality of cells derived from a human induced pluripotent stem cells (iPSC) lines, wherein somatic mutations M415V and/or M415I in the gene USP48 have been introduced into certain cells of the organoid.

Item 13. The organoid of Item 12, wherein the somatic mutations M415V and/or M415I are introduced by CRISPR.

Item 14. A method of screening for compounds that have therapeutic effects for Cushing's Disease (CD) in human, comprising
a) contacting the organoid of any of Items 1-7 with one or more compounds, and
b) assessing apoptosis or hormonal secretion profile of the organoid after the contacting step (a),
c) identifying a candidate compound that induces apoptosis or reduces ACTH secretion.

Item 15. A method of screening for compounds that have therapeutic effects for Cushing's Disease (CD) in human, comprising
a) contacting the organoid of any of Items 12-13 with one or more compounds, and
b) assessing apoptosis or hormonal secretion profile of the organoid after the contacting step (a),
c) identifying a candidate compound that induces apoptosis or reduces ACTH secretion.

All references cited in this disclosure, including but not limited to patents, patent applications and published papers, are hereby incorporated by reference into this disclosure.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 Materials and Methods

### 1.1. Generation and Culture of Human Pituitary Neuroendocrine Tumor (PitNET) Organoids

Patients with planned transsphenoidal surgery for pituitary tumors were identified in the outpatient neurosurgery clinics. Tissues were collected under the St. Joseph's Hospital and Barrow Neurological Institute Biobank collection protocol PHXA-05TS038 and collection of outcomes data protocol PHXA-0004-72-29, with the approval of the Institutional Review Board (IRB) and patient consent. Samples were de-identified and shipped to the Zavros laboratory (University of Arizona) for processing.

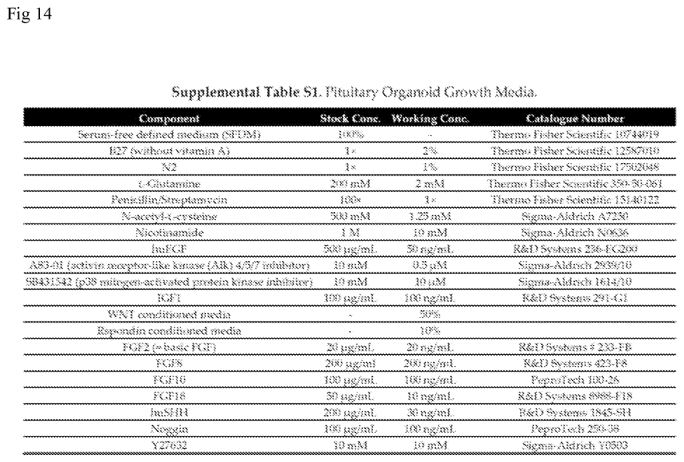

Pituitary adenoma tissue was collected in Serum-Free Defined Medium (SFDM) supplemented with ROCK inhibitor (Y27632, 10 UM), L-glutamine (2 mM), A83-01 (activin receptor-like kinase (Alk) 4/5/7 inhibitor, 0.5 mM), penicillin/streptavidin (1%), kanamycin (1%), amphotericin/gentamycin (0.2%), CHIR-98014 (4 mM), and thiazovivin (TZV, 2.5 mM). Tissues that contained red blood cells were incubated with Red Blood Cell (RBC) Lysis Buffer according to the manufacturer's protocol (Thermo Fisher Scientific). Tissues were dissected into small pieces, transferred to digestion buffer (DMEM/F12 supplemented with 0.4% collagenase 2, 0.1% hyaluronic acid, 0.03% tryp-sin-EDTA) and incubated for 5-10 min at 37° C. with gentle shaking. Tissue was further incubated with Accutase™ (Thermo Fisher Scientific) for 5 min at 37° C. Enzymatically dissociated cells were pelleted and washed using in DPBS supplemented with antibiotics at a 400 relative centrifugal force (RCF) for 5 min. Dissociated adenoma cells were re-suspended in Matrigel™ and Matrigel™ domes containing the cells were plated in cul-ture dishes and overlaid with pituitary growth media (FIG. 14). The culture was maintained at 37° C. at a relative humidity of 95% and 5% CO₂. Organoid growth media was replenished every 3-4 days and passaged after 15 days in culture.

1.2. Generation of Induced Pluripotent Stem Cells (iPSCs)

Induced pluripotent stem cell lines (iPSC lines) were generated from control individuals (no reported disease) or CD patients according to published protocols by the University of Arizona iPSC Core [26]. All human iPSC lines were tested to be negative for myco-plasma contamination using the Mycoalert Mycoplasma testing kits (LT07-318, Lonza) and no karyotype abnormalities were found (KaryoStat+, Thermo).

1.3. Pituitary Organoids Generated from iPSCs

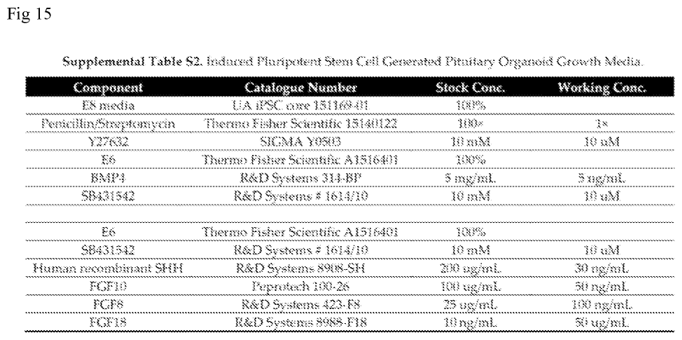

Six well culture plates were coated with 2 mL/well 0.67% Matrigel (diluted in E8 media, UA iPSC core, 151169-01) and incubated at 37° C. at a relative humidity of 95% and 5% CO₂ overnight. The iPSC lines were reprogrammed from either a healthy donor (JCAZ001) or, CD patient (iPSC⁷ and iPSC¹⁰⁶³) blood at the University of Arizona iPSC Core. Passage 12 iPSCs were plated onto the coated plates and incubated at 37° C. at a relative humidity of 95% and 5% CO₂. At 70% confluency, cells were passaged to a freshly coated 24 well plates at a ratio of 1:8 and grown to 85-90% confluency before beginning the directed differentiation schedule. From days 0 to 3, cells were cultured in E6 media supplemented with 1% penicillin/streptomycin, 10 uM SB431542 and 5 ng/ml BMP4. BMP4 was withdrawn from the culture at day 3. Starting on day 4, the cells were cultured in E6 media, supplemented with 10 UM SB431542, 30 ng/ml human recombinant SHH, 100 ng/ml FGF8b, 10 ng/ml FGF18 and 50 ng/ml FGF10. Fifteen days after culture, the cells were harvested in cold E6 media by pipetting and resuspended in Matrigel™ (20,000 cells/50 ml Matrigel™). Matrigel™ domes containing the cells were plated in culture dishes and overlaid with differentiation media containing E6 media supplemented with 10 uM Y-27632, 30 ng/ml human recombinant SHH, 100 ng/ml FGF8b, 10 ng/ml FGF 18 and 50 ng/ml FGF10 (FIG. 15). Organoids were cultured for a further 15 days at 37° C. at a relative humidity of 95% and 5% CO₂

### 1.4. Spectral Flow Cytometry (Cytek™ Aurora)

The multicolor flow cytometry panel was designed using the Cytek®Full Spectrum Viewer online tool to calculate the similarity index. The organoids were harvested in cold SFDM media and centrifuged at 400×g for 5 minutes. Supernatant was discarded and organoids were dissociated to single cells using Accutase® (Thermo Fisher Scientific 00-4555-56). The enzymatic reaction was stopped using prewarmed DPBS and cells were then centrifuged at 400×g for 5 minutes and incubated with fluorochrome-conjugated/unconjugated primary surface or cytoplasmic antibodies at 4° C. for 30 minutes. Cells were then washed with Cell Staining Buffer (BioLegend #420-201) and incubated with secondary antibodies at 4° C. for 30 minutes. Cells were fixed using Cytofix/Cytoperm™ Fixation/Permeabilization Solution (BD Biosciences #554714) at 4° C. for 20 minutes, followed by washing with Fixation/Permeabilization wash buffer. Cells were then labeled with fluorochrome-conjugated/unconjugated intracellular primary antibodies at 4° C. for 30 minutes, washed and incubated with secondary antibodies at 4° C. for 30 minutes. Cells resuspended in cell staining buffer and fluorescence measured using the Cytek Aurora 5 Laser Spectral Flow Cytometer. An unstained cell sample was fixed and used as reference control. UltraComp eBeads™, Compensation Beads (Thermo Fisher Scientific #01-2222-42) were stained with the individual antibodies and used as single stain control for compensation and gating. Data was acquired using the Cytek™ Aurora and analyzed using Cytobank software (Beckman Coulter, Indianapolis, IN).

### 1.5. Whole Mount Immunofluorescence

Organoids were immunostained using published protocols by our laboratory [27-29]. Proliferation was measured by using 5-ethynyl-2′-deoxyuridine (EdU) incorporation according to the Manufacturer's protocol (Click-IT EdU Alexa Fluor 555 Imaging Kit, Thermo Fisher Scientific C10338). Costaining was performed by blocking fixed organoids with 2% donkey serum (Jackson Immuno Research, #017-000-121) diluted in 0.01% PBST for 1 hr. at room temperature. Organoids were then incubated overnight at 4° C. with primary antibodies followed by secondary antibodies and Hoechst (Thermo Fisher Scientific H1399, 1:1000 in 0.01% PBST) for 1 hr at room temperature. Human specific primary antibodies used included: rabbit anti-ACTH (Thermo Fisher Scientific 701293, 1:250), rabbit anti-Synaptophysin (Thermo Fisher Scientific PA5-27286, 1:100), species PIT1 (Thermo Fisher Scientific PA5-98650, 1:50), rabbit anti-LH (Thermo Fisher Scientific PA5-102674, 1:100), mouse anti-FSH (Thermo Fisher Scientific MIF2709, 1:100), mouse anti-PRL (Thermo Fisher Scientific CF500720, 1:100), Alexa Flour conjugated GH (NB500-364AF647, 1:100) and mouse anti-CAM5.2 (SIGMA 452M-95, 1:250). The secondary antibodies used included Alexa Fluor 488 Donkey Anti Rabbit IgG (H+L) (Thermo Fisher Scientific A21206, 1:100) or Alexa Fluor 647 Donkey Anti Mouse IgG (H+L) (Thermo Fisher Scientific A31571, 1:100). Organoids were visualized and images acquired by confocal microscopy using the Nikon CrestV2 Spinning Disk. Fluorescence intensity and percentage of EdU positive cells of total cells, were calculated using Nikon Elements Software (Version 5.21.05).

### 1.6. Nuclear Morphometric Analysis (NMA)

Nuclear Morphometric Analysis (NMA) using treated organoids was performed based on a published protocol that measures cell viability based on the changes in nuclear morphology of the cells using nuclear stain Hoechst or DAPI [30]. Images of organoid nuclei were analyzed using the ImageJ Nuclear Irregularity Index (NII) plugin for key parameters that included cell area, radius ratio, area box, aspect, and roundness. Using the published spreadsheet template [30], the NII of each cell was calculated with the following formula: NII=Aspect−Area Box+Radius Ratio+Roundness. The area vs NII of vehicle-treated cells were plotted as a scatter plot using the template and considered as the normal cell nuclei. The same plots were generated for each condition, and the NII and area of treated cells were compared to the normal nuclei, and classified as one of the following NMA populations: Normal (N; similar area and NII), Mitotic (S; similar area, slightly higher NII), Irregular (I; similar area, high NII), Small Regular (SR; apoptotic, low area and NII), Senescent (LR; high area, low NII), Small Irregular (SI; low area, high NII), or Large Irregular (LI; high area, high NII). Cells classified as SR exhibited early stages of apoptosis, and cells classified as either I, SI or LI exhibited significant nuclear damage. The percentage of cells in each NII classification category were calculated and plotted as a histogram using GraphPad Prism.

### 1.7. ELISA

Concentration of secreted ACTH in conditioned media that was collected from organoid cultures was measured using the Human ACTH ELISA Kit (Novus Biologicals, NBP2-66401) according to the manufacturer's protocol. The enzyme-substrate reaction was measured spectrophotometrically (BioTek Gen5 Micro Plate Reader Version 3.11) at a wavelength of 450 nm and the ACTH concentration (pg/mL) was interpolated using a standard curve with a 4-parameter logistic regression analysis using GraphPad Prism (Version 9.2.0).

### 1.8. Drug Assay

Patient adenoma-derived pituitary organoids were grown in 96-well plates and treated with 147 small molecules taken from the NCI AOD9 compound library for 72 hrs. (https://dtp.cancer.gov/organization/dscb/obtaining/available_plates.html). Drugs were diluted from 10 mM DMSO stock plates into 100 uM DMSO working stocks with a final concentration of 1 uM. All vehicle controls were treated with 0.1% DMSO. Organoid proliferation was measured using CellTiter 96® AQueous One Solution Cell Proliferation Assay kit (MTS, Promega, G3582) according to the manufacturer's instruction. Organoid death was calculated based on the absorbance readings at 490 nm, collected from the MTS assay relative to the vehicle controls. Drug screens were performed with biological replicates in same screen. Drugs were selected based on their ability to target key signaling pathways and clinical relevance to the treatment. Drug sensitivity is represented by cell viability and is significant at <0.5 suppressive effect of the drugs. The percent cell viability relative to the vehicle control was calculated. Correlation coefficient across each organoid were calculated using Pearson method to assess confidence in replication. Variance Component was detected for each drug across all organoids. A random effect model was run with a single random factor for each drug and estimated variance was calculated by rejecting the null hypothesis that there isn't variation among samples. The drug responses were grouped by variance factor large (vc>100), median (100>vc>50), and small (vc<50). Heatmap was used to display the differential responses in cell viability for the drugs.

Drugs that clustered together and showed response within corticotrophs were investigated further based on their mode of action. Pathways (Kegg and Reactome) and gene ontology mapping was conducted for the genes that were being targeted by the drugs to evaluate the key responses in cellular processes. Network was constructed in Cytoscape v 3.8.2 for association between the drugs and genes.

### 1.9. Drug Dose Responses

Organoids were grown in Matrigel™ domes within 96-well round-bottom culture plates. Recombinant human SHH was removed from the pituitary organoid growth media, 24 hrs. prior to drug treatment. Organoids were treated with either vehicle (DMSO), cabergoline (Selleckchem S5842), ketoconazole (Selleckchem S1353), roscovitine (Selleckchem S1153), GANT61 (Stemcell Technologies 73692), pasireotide (TargetMol TP2207), mifeprostone (Selleckchem S2606), etomidate (Selleckchem S1329), mitotane (Selleckchem S1732), metyropane (Selleckchem S5416), or osilodrostat (Selleckchem S7456) at concentrations of 0, 1, 10, 100, 1000, and 10000 nM for 72 hours. Percent cell viability was measured using an MTS assay (Promega G3580). Absorbance was measured at 490 nm and normalized to the vehicle. Concentrations were plotted in a logarithmic scale, and a nonlinear dose-response curve regression was calculated using GraphPad Prism. An IC50 value for each drug treatment was determined based on the dose response curve using GraphPad Prism analysis software.

### 1.10. AUC Method Needs to Add in Dose Response

AUC (Area under the curve) was determined by plotting the normalized % cell viability versus transformed concentration of the drugs using a trapezoidal approximation for the area [31]. The formula was based on splitting the curve into trapezoids with bases equal to the % viability (V) and height equal to the interval length (difference in concentrations, C) and summing the areas of each trapezoid:

$${\sum }_{0}^{n} \frac{( {V}_{n} + {V}_{n - 1} )}{2} * ( {C}_{n} - {C}_{n - 1} )$$

1.11. Quantitative RT PCR (qRT-PCR)

RNA was collected from patient-derived organoid cultures using the RNeasy Mini Kit (Qiagen). cDNA was generated from the extracted RNA, and then pre-amplified using TaqMan PreAmp Master Mix (Thermo Fisher Scientific 391128). The primers used were human-specific GAPDH (Thermo Fisher Scientific, Applied Biosystems Hs02786624_g1), NR5A1 (SF1) (Thermo Fisher Scientific, Hs00610436_m1), PIT1 (Thermo Fisher Scientific, Hs00230821_m1), TPit (Thermo Fisher Scientific, Hs00193027) and POMC (Thermo Fisher Scientific, Hs01596743_m1). Each PCR reaction was performed using a final volume of 20 μL, composed of 20× TaqMan Expression Assay primers, 2× TaqMan Universal Master Mix (Applied Biosystems, TaqMan® Gene Expression Systems) and a cDNA template. Amplification of each PCR reaction was conducted in StepOne™ Real-Time PCR System (Applied Biosystems), using the following PCR conditions: 2 minutes at 50° C., 10 minutes at 95° C., denaturing for 15 seconds at 95° C. and annealing/extending for 1 minute at 60° C., for a total of 40 cycles. Relative fold change was calculated using the 2−ΔΔCt method [32], where CT=threshold cycle. Results were analyzed as the average fold change in gene expression compared to the control, and GAPDH served as an internal control.

### 1.12. Whole Exome Sequencing

WES was performed by the University of Arizona Center for Applied Genetics and Genomic Medicine. Isolated DNA from patient adenoma tissue will be quantified using the Qubit quantitation system with standard curve as per the supplier protocol (Thermo Fisher Scientific) and all samples were further tested for quality using the Fragment Analyzer (Advanced Analytical) following the manufacturer-recommended protocols. Whole exome sequencing (WES) was performed by array capture of and approximately 60 Mb of exome target sequence using the SureSelectXT Human All Exon V6 enrichment (Agilent) or equivalent (which one was used). All exome library builds were quantified via qPCR and subsequently sequenced to a minimum 20× coverage using paired-end chemistry on the Illumina NovaSeq platform. Whole exome sequencing (WES) was performed by hybridization capture of approx. 35 Mb of exome target sequence using the Swift Exome Hyb Panel (Swift Biosciences 83216). All exome library builds were quantified via qPCR and subsequently sequenced to a minimum 20× coverage using paired-end chemistry on the Illumina NextSeq500 or NovaSeq platform (Illumina). DNA reads were trimmed, filtered by quality scores and aligned to human genome (hg38) with Burrows-Wheeler Aligner with default parameters. Picard (http://broadinstitute.github.io/picard) was used to mark duplicates. Germline single nucleotide variants (SNV) were called using the Genome Analysis Tool Kit (GATK), using the given guidelines. Mutations were annotated using ANNOVAR for coding sequences. Variants that passed the quality filter were further investigated for similarity. Concordance between Tissue and Organoids was calculated using Jaccard similarity index (Jᵢⱼ=Mᵢⱼ/(Mᵢ+Mⱼ−Mᵢⱼ) where Mᵢ is the number of variants in Tissues, Mⱼ is the number of variants in Organoids and Mᵢⱼ is the number of identical variants in both tissue and organoid.

1.13. Single Cell RNA Sequencing (scRNA-Seq)

Cultures were collected on day 15 of the pituitary directed differentiation schedule and cells were dissociated into a single-cell suspension using Cell Dissociation Buffer (Thermo Fisher Scientific 13151014). Cells (15,000 cells/sample) were resuspended in sample buffer (BD Biosciences 65000062), filtered using cell strainer (40 microns) and loaded into a BD Rhapsody cartridge (BD Biosciences 400000847) for single-cell transcriptome isolation. Based on the BD Rhapsody system whole-transcriptome analysis for single-cell whole-transcriptome analysis, microbead-captured single-cell transcriptomes were used to prepare a cDNA library. Briefly, double-stranded cDNA was first generated from the microbead-captured single-cell transcriptome in several steps, including reverse transcription, second-strand synthesis, end preparation, adapter ligation, and whole-transcriptome amplification (WTA). Then, the final cDNA library was generated from double-stranded full-length cDNA by random priming amplification using a BD Rhapsody cDNA Kit (BD Biosciences, 633773) and the BD Rhapsody Targeted mRNA and WTA Amplification Kit (BD Biosciences, 633801). The library was sequenced in PE150 mode (paired-end with 150-bp reads) on NovaSeq6000 System (Illumina). 80,000 reads were demultiplexed, trimmed, mapped to the GRCh38 annotation, and quantified using the whole transcriptome analysis pipeline (BD Rhapsody™ WTA Analysis Pipeline v1.10 rev6) on the Seven Bridges Genomics platform (https://igor.sbgenomics.com), prior to clustering analysis in Seurat. For QC & filtration, read counting and unique molecular identifier (UMI) counting were the principal gene expression quantification schemes used in this single-cell RNA-sequencing (scRNA-seq) analysis. The low-quality cells, empty droplets, cell doublets or multiplets were excluded based on unique feature count (less than 200 or larger than 2500) as they may often exhibit either an aberrantly high gene count or very few genes. Additionally, the mitochondrial QC metrics was calculated, and the cells with >5% mitochondrial counts were filtered out, because the percentage of counts originating from a set of low-quality or dying cells often exhibit extensive mitochondrial contamination. After the removal of unwanted cells from the single cell dataset, a global-scaling normalization method LogNormalize was employed which normalizes the feature expression measurements for each cell by the total expression, multiplies this by a scale factor (10,000) and log-transforms the result. The molecules per gene per cell based on RSEC error correction (RSEC_MolsPerCell file) matrix files from iPSC^{ctrl} and iPSC^{CDH23} samples were imported into Seurat v4, merged, and processed (as stated above) for UMAP reduction, cluster identification, and differential marker assessment using the FindAllMarkers function within Seurat.

### 1.14. Statistical Analyses

Sample size was based on assessment of power analysis using SigmaStat software. Data collected from each study from at least 4 in vitro technical replicates unless otherwise stated was analyzed by obtaining the mean±standard error of the mean (SEM) and significance of the results were then tested using commercially available software (GraphPad Prism, GraphPad software, San Diego, CA.

### Example 2 Generation and Validation of Human Pituitary Adenoma-Derived Organoids

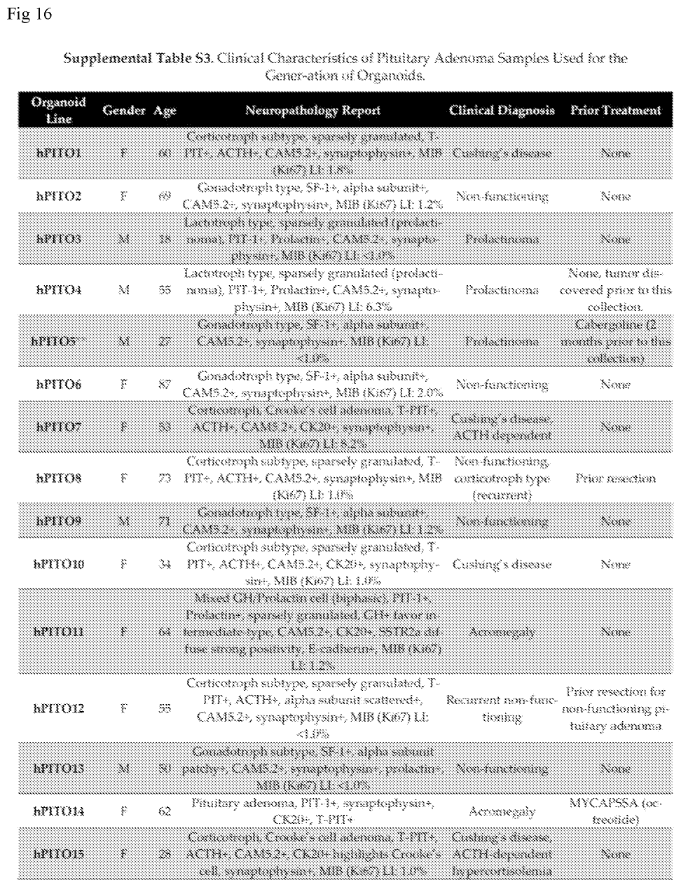

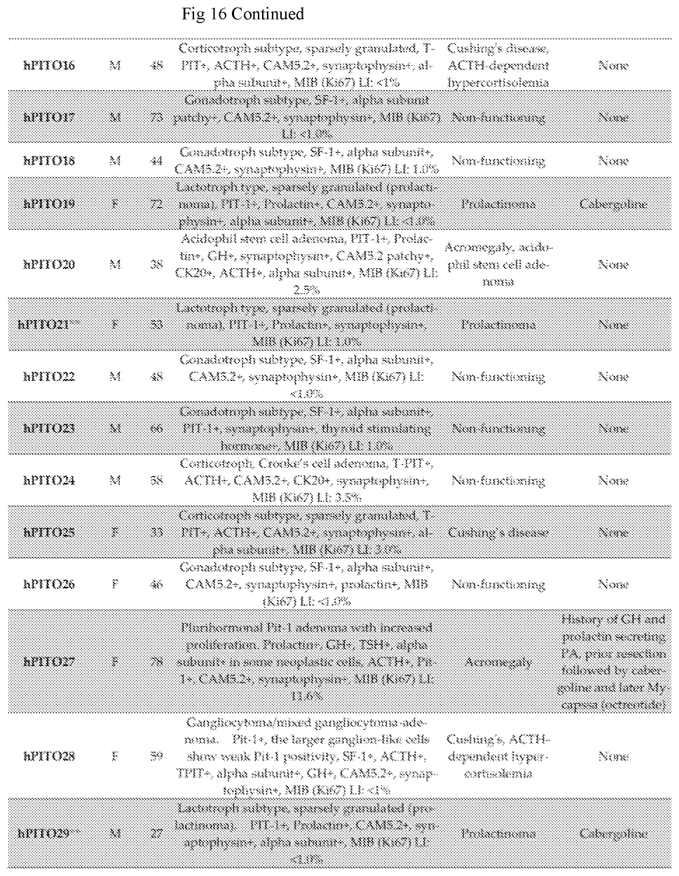

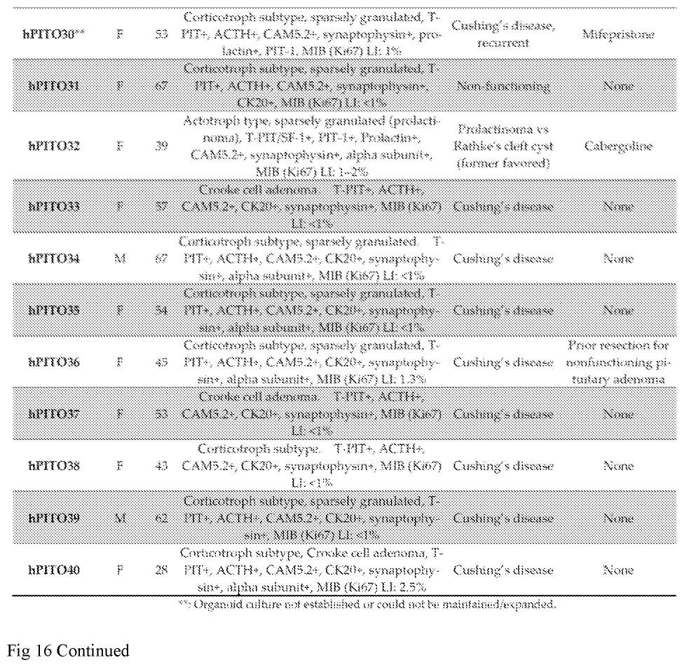

Human adenoma tissue was harvested during endoscopic transsphenoidal pituitary surgery from 35 patients to generate organoids. These cultures are referred to as human pituitary adenoma derived organoids (hPITOs). In FIG. 16, Supplementary Table 3 summarizes the neuropathology reports and clinical diagnosis from these cases. In summary, 12 corticotroph (functional, CD), and 3 silent corticotroph adenomas (nonfunctional adenomas), 9 gonadotroph adenomas, 8 lactotroph adenomas, and 3 somatotroph adenomas (acromegaly) were used to generate hPITOs (FIG. 16).

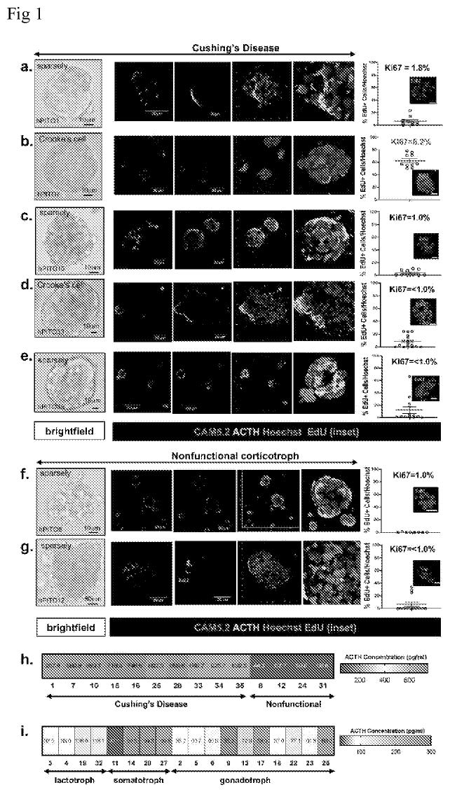

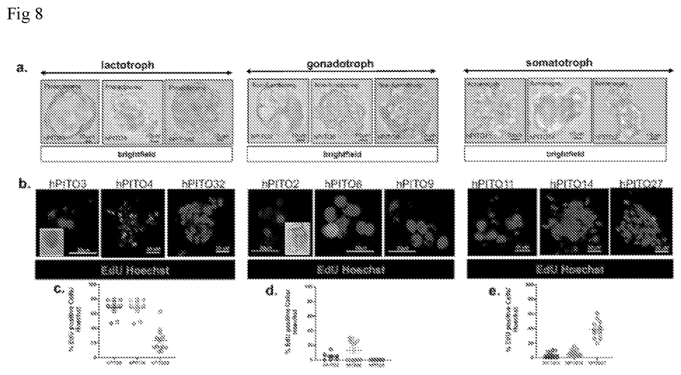

Bright-field microscopy images of hPITOs that were generated from corticotroph adenomas from patients diagnosed with CD (FIG. 1a-e) and silent/nonfunctioning tumors (FIG. 1f, g), revealed morphological diversity among the organoid lines between individual patients and amongst subtypes. Confocal microscopy was used to capture a z-stack through the hPITO38 immunofluorescently stained for CAM5.2 (red), ACTH (green) and Hoechst (nuclear staining, blue) and emphasizes the 3D cellular structure of the hPITOs. Lactotroph, gonadotroph and somatotroph adenomas were used to generate hPITOs and showed the same morphological divergence amongst subtypes and between each patient line (FIG. 8). Proliferation was measured within the cultures using 5-ethynyl-2′-deoxyuridine (EdU) uptake and showed that the percentage of EdU+ve cells/total Hoechst+ve nuclei directly correlated with the pathology MIB-1 (Ki67) score (red, R2=0.9256) (FIG. 1a-g, FIG. 8). ACTH concentration, that was measured by ELISA using organoid conditioned culture media collected from each hPITO line, showed highest expression in the corticotroph adenoma organoids generated from CD patients (FIG. 1h).

### Example 3 Characterization of Cell Lineages in Pituitary Adenoma-Derived Organoids by Spectral Cytek™ Aurora Analysis

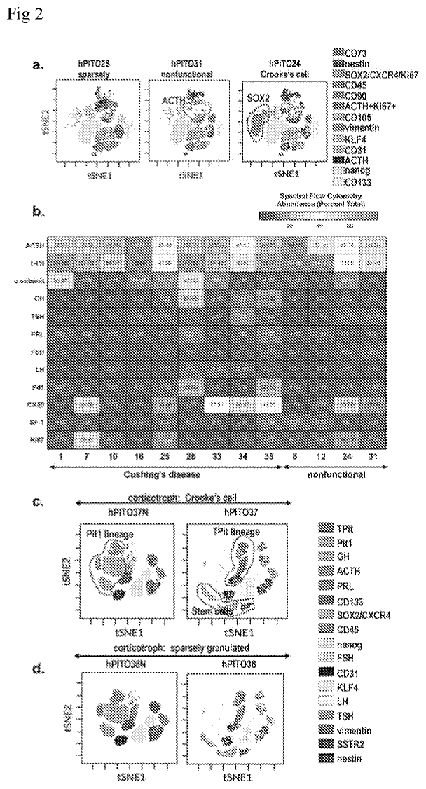

To validate the similarity in cell lineages identified between the organoid line and the patient's tumor, we compared the immunohistochemistry from the neuropathology report (FIG. 16) to the expression pattern of pituitary adenoma specific markers that were measured using Cytek™ Aurora spectral flow cytometry (FIG. 2). The location of cells that are found in each cluster based on the highly expressed antigens are shown in the representative tSNE (viSNE) maps (FIG. 2a). Compared to nonfunctional adenoma-derived hPITOs, organoids derived from corticotroph adenomas of CD patients highly expressed proliferating (Ki67+) T-Pit+ACTH cells (FIG. 2a). Interestingly, there was an increase in SOX2+ cells within the total cell population associated with Crooke's cell adenoma hPITOs (FIG. 2a). Within the total cells population cell clusters expressing CD45 and vimentin were also measured (FIG. 2a). Data for the analysis of corticotroph hPITOs derived from CD patients and individuals with nonfunctional adenomas was summarized in a heatmap for each subtype organoid line based on quantified cell abundance (percent of total cells) using spectral flow cytometry (FIG. 2b).

Organoid cultures derived from pituitary adenomas (hPITO37 and hPITO38) were compared to organoids derived from adjacent normal pituitary tissue (hPITO37N and hPITO38N) (FIG. 2c, d). While Pit1 lineages including cells expressing GH and PRL, and SF1 lineage expressing FSH and LH, were detected in the hPITO37N and hPITO38N organoid cultures, these cell populations were significantly reduced within the patient's matched adenoma tissue (FIG. 2c, d). Overall, hPITOs derived from CD patients expressed increased stem and progenitor cell markers including CXCR4, SOX2, and CD133 (FIG. 2). Collectively, our findings of the characterization of the hPITO cultures support our prediction that this in vitro model recapitulates much of the patient's adenoma pathophysiology.

### Example 4. Inherent Patient Differences to Drug Response is Reflected in the Organoid Culture

Tumor recurrence can occur in as many as 30-50% of CD patients after successful surgical treatment [10,33,34]. Unfortunately, bilateral adrenalectomy is the chosen surgical treatment for patients with persistent CD [35]. Bilateral adrenalectomy leads to the increased risk for development of Nelson's syndrome (progressive hyperpigmentation due to ACTH secretion and expansion of the residual pituitary tumor). Although the risk of developing Nelson's syndrome following adrenalectomy can be reduced by 50% with stereotactic radiotherapy [35], there is a need to develop medical therapies that directly target the pituitary adenoma. Thus, we established a high-throughput drug screening assay using patient-derived pituitary organoids. After 72 hours of treatment, cell viability was measured using an MTS assay and data was represented as a heatmap whereby blue indicates higher cell death, and red suggests higher cell viability. The replicates behaved consistently with the drug response with the correlation scores of >0.8 for these samples (FIG. 3a). We estimated the variance component for each drug across all organoids. Variation among samples were found to be significant (P=<0.05) for each of the 83 drugs. The drug responses were grouped by variance factor large, median, and small. The larger the variance the more variable is the drug response across the organoids. We noted a set of drugs that showed significant differential response across the functional corticotroph organoids. Unsupervised clustering of drug responses across organoids shows a pattern that relates to our statistically calculated results (FIG. 3a, c), and the replicates for each independent organoid cluster together. The drugs with higher variance component across all the functional corticotrophs cluster together as a group (FIG. 3a). These drugs show cell viability of 10% to 60% across different organoids. Analyzing the pattern more closely we observe that, within a pathologically defined group, there was differential organoid response to drugs and there were inherent patient differences to drugs within this group. FIG. 3 demonstrates a variation in drug responsiveness amongst the organoid lines generated from individual patients. Importantly, there was further divergence in drug responsiveness amongst the individual organoid lines within each pathologically defined corticotroph subtype. These data clearly demonstrate that the inherent patient difference to drug response that is often observed among CD patients is reflected in the organoid culture.

Drugs that clustered together and showed correlated responses were investigated further for their mode of action based on target genes (FIG. 3d). The genes were analyzed for their associations in cellular pathways and gene ontology functional processes. Identified drug-gene pairs were interconnected by cellular pathways that are known to regulate cell cycle, WNT signaling, hedgehog signaling and neuroactive ligand-receptor interaction signaling pathways (FIG. 3d). These identified genes were influence multiple cellular functions such as cytokine-cytokine receptor interactions and Notch signaling. Proteosome 20S subunit genes PSMAs/PSMBs and the HDAC gene family are involved in many cellular functions. The ephrin receptors (EPHs), adrenoceptor alpha receptors (ADRs), dopamine receptors (DRDs) and the 5-hydroxytryptamine serotonin receptors (HTRs) gene families influence neuronal functions and are targeted by multiple drugs in our focused cluster. Thus, FIG. 3d suggests that there are complex connections of genes and processes effected in response to the drugs. These data reveal potential therapeutic pathways for CD patients.

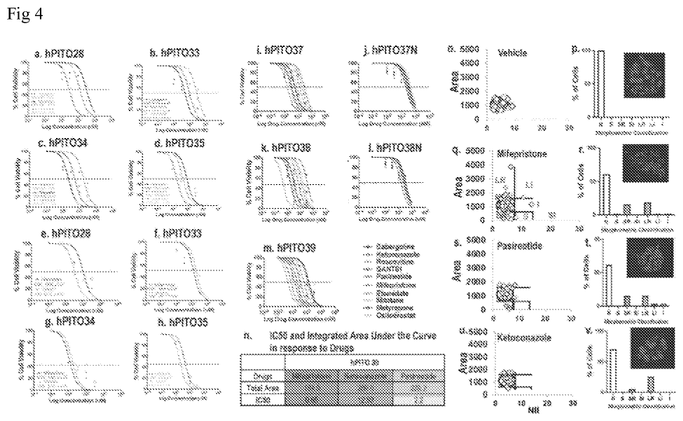

Divergent half maximal inhibitory concentration (IC50) values, as documented by an MTS cell viability assay, were observed in response to drug treatment among hPITOs lines 28, 33, 34, 35 and 37. Note that a shift of the curve to the right indicates a higher IC50 (ie, more resistant to that drug). Cell viability assays were normalized to vehicle-treated controls to ensure that toxicity was specific o the drug effects (FIG. 4). Dose response curves for organoid 33 and organoid 34 shows better response at lower dose for cabergoline compared to Metyrapone and osilodrostat but different for organoid 35 where Metyrapone and osilodrostat gave better responses than Cabergoline (FIG. 4a-h). For drugs mifepristone and GANT61, 33 and 34 have same level of response to both the drugs but when the two organoid responses are compared 34 had better response than 33 (FIG. 4a-h). Similar divergent drug responses were observed in hPITO lines 37 and 38 (FIGS. 4i and k). However, organoids generated from adjacent normal pituitary tissue from patients 37 and 38 were nonresponsive to the same standard of care or investigational drugs for CD (FIGS. 4j and l). These data were consistent with observation made in the drug screen (FIG. 3a-c) and demonstrate that there was an inherent difference to drug response within the organoid cultures of the same corticotroph subtype.

In addition to cell viability, Nuclear Morphometric Analysis (NMA) using treated organoids was performed based on a published protocol that measures cell viability based on the changes in nuclear morphology of the cells using nuclear stain Hoechst or DAPI [30]. Nuclear Irregularity Index (NII) was measured based on the quantification of the morphometric changes in the nuclei in response to standard-of-care drugs mifepristone, pasireotide and ketoconazole in hPITO39 (FIGS. 4o-v). The area vs NII of vehicle-treated cells were plotted as a scatter plot using the template and considered as the normal cell nuclei (FIG. 4o). The same plots were generated for mifepristone (FIG. 4q), pasireotide (FIG. 4s), and ketoconazole (FIG. 4u). The NII and area of treated cells were compared to the normal nuclei, and classified as one of the following NMA populations: Normal (N; similar area and NII), Mitotic (S; similar area, slightly higher NII), Irregular (I; similar area, high NII), Small Regular (SR; apoptotic, low area and NII), Senescent (LR; high area, low NII), Small Irregular (SI; low area, high NII), or Large Irregular (LI; high area, high NII) (FIGS. 4p, 4r, 4t, 4v). Cells classified as SR exhibited early stages of apoptosis, and cells classified as either I, SI or LI exhibited significant nuclear damage. Data showed that mifepristone induced significant apoptosis in hPITO39 cultures (FIG. 4r) compared to responses to pasireotide (FIG. 4t), and ketoconazole (FIG. 4v). These responses were consistent with the IC50 and total area under the curve in response to drugs (FIG. 4m, 4n). Measurement of NII is an approach may be used to confirm potential drug targets identified from the drug screen.

### Example 5 Organoid Responsiveness to Pasireotide Correlates with SSTR2 and SSTR5 Expression

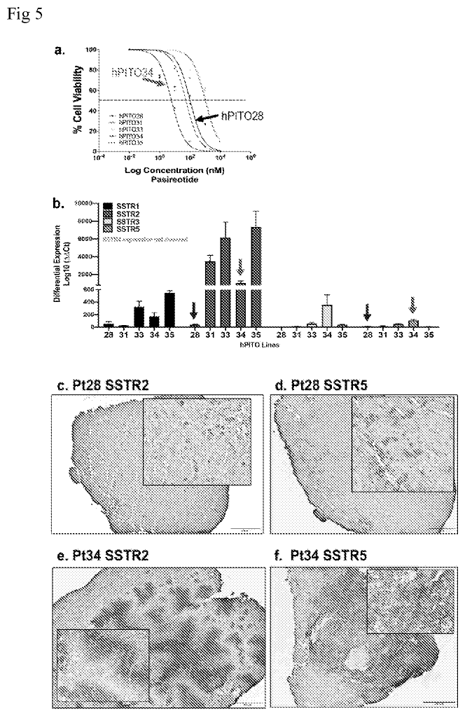

Organoid lines hPITO28, 31, 33, 34 and 35 exhibited divergent IC50 values in response to SSTR agonist pasireotide (FIG. 5a). hPITO34 was the most responsive to pasireotide, with a low IC50 value of 6.1 nM (FIG. 5a). Organoid lines hPITO33 and hPITO35 were least responsive with IC50 values of 1.2 UM and 1 uM respectively in response to pasireotide (FIG. 5a). The expression of SSTR subtypes 1-5 among the different organoid lines were measured by qRT-PCR and IHC (FIG. 5b). One of the least responsive organoid lines, hPITO28, exhibited lower differential expression in SSTR2 and SSTR5 compared to the highly responsive hPITO34 line (FIGS. 5a, 5b). Gene expression levels of SSTR2 and SSTR5 within hPITO28 and 34 correlated with protein levels within the patient's tumor tissue (FIGS. 5c-f). Given the greater binding affinity for SSTR5 compared to SSTR2 by pasireotide, these data were consistent with greater responsiveness to drug by hPITO34 in comparison to hPITO28 (FIGS. 5a, 5c-f). In addition, the expression of SSTR subtypes 2 and 5 within the organoid cultures correlated with the expression patterns of the patient's tumor tissues (FIGS. 5a, 5c-f).

### Example 6 Organoids Derived from Pituitary Corticotroph Adenomas Retain the Genetic Alterations of the Patient's Primary Tumor

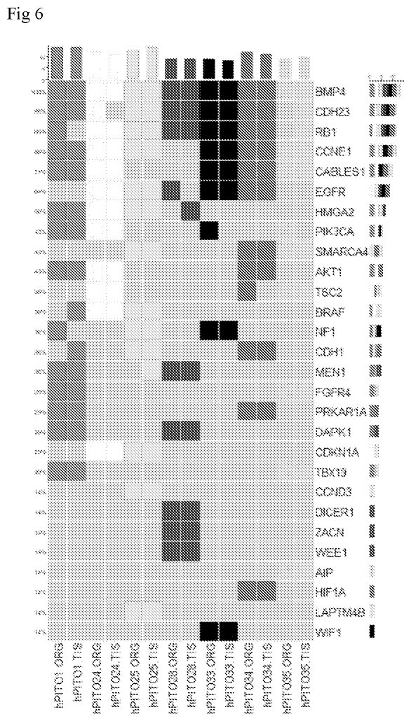

To identify the genetic features of the organoids derived from pituitary adenomas of CD patients, whole-exome sequencing (WES) of hPITOs and the corresponding primary adenoma tissues was performed. WES analysis of each hPITO line was performed and the results compared with those for the corresponding primary adenoma tissues. It is shown that the concordance rate of exonic variants between the primary tumor tissues obtained from CD patients and the corresponding organoid line. On an average approximately 5000 mutations across each of the 14 paired samples of organoids and tissues were identified. For the variants detected all 7 pairs showed Jaccard index ranging from 0.5 to 0.8. Out of 7 pairs, 5 (hPITO24, 25, 28 and 35) pairs had Jaccard score of 0.8 while hPITO33 and 34 pairs had 0.7 and hPITO1 had 0.5. To investigate the similarity across the SNV (single nucleotide variation) sites, we calculated Jaccard index of exon sites for synonymous and non-synonymous events and found scores for all pairs ranging from 0.8 to 0.9 and further for only non-synonymous events Jaccard scores were also from 0.8 to 0.9, except for hPITO1 which showed overall lower concordance and a score of 0.4 to 0.5. FIG. 6 shows non-synonymous mutations found in organoid and tissue pairs for some of the key genes that are known to be involved in pituitary adenoma disease. Concordance indices between organoids and matched patient's adenoma tissues is reported in FIG. 6. Therefore, WES data demonstrated that organoids derived from pituitary corticotroph adenomas retained the genetic alterations of the patient's primary tumor tissue.

### Example 7 IPSC Pituitary Organoids Generated from a CD Patients Expressing Familial Mutations Reveal Corticotroph Adenoma Pathology In Vitro

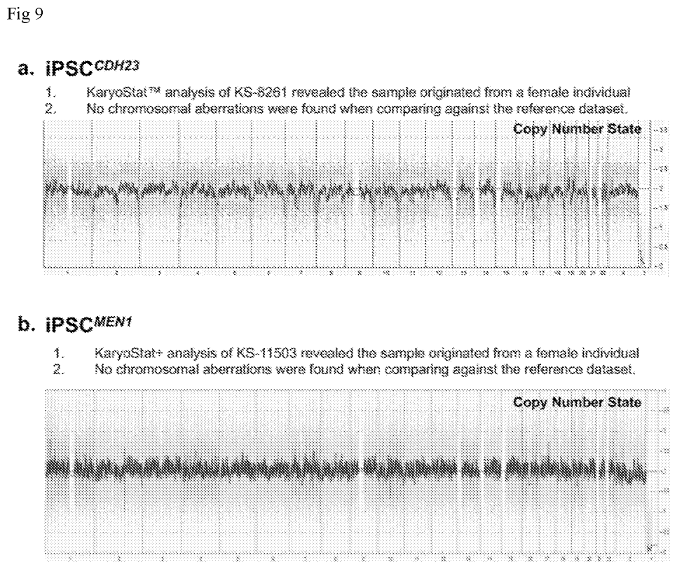

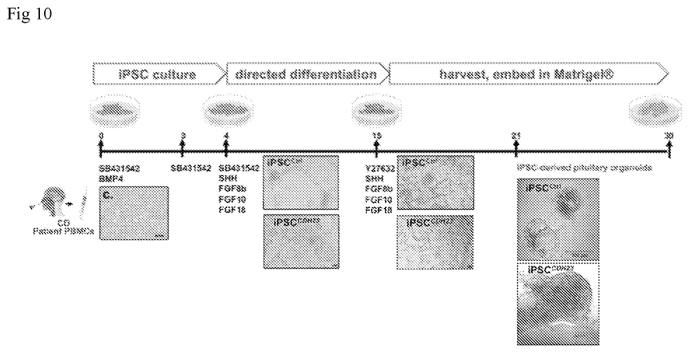

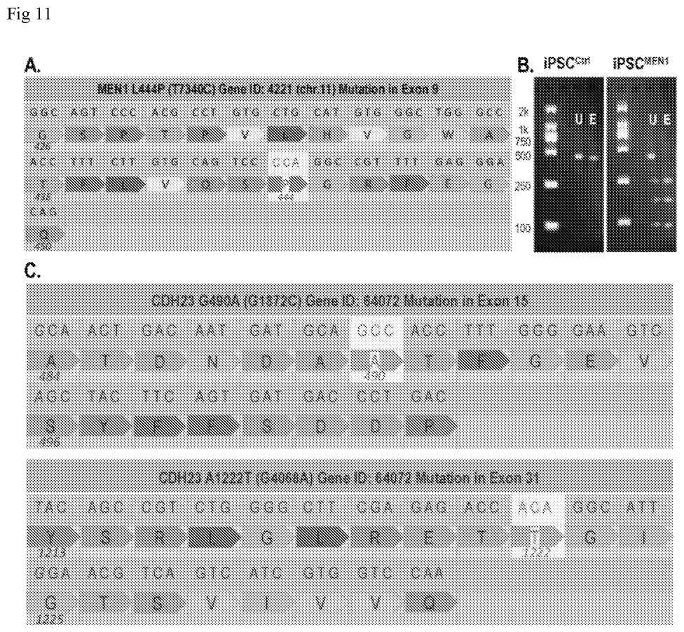

Extensive research has revealed the role of somatic and germline mutations in the development of CD adenomas [36,37]. Pituitary organoids were developed from iPSCs generated from the PBMCs of CD patients and carrying germline mutations that were identified by WES (FIG. 10). Chromosomal aberrations were not found when comparing against the reference dataset in the iPSCs generated from the CD patients (FIGS. 9a, 9b). PBMCs isolated from patients diagnosed with CD were analyzed by WES to determine the expression of germline mutations. WES revealed the expression of a more recently identified gene predisposing patients to CD, cadherin-related 23 (FIG. 11).

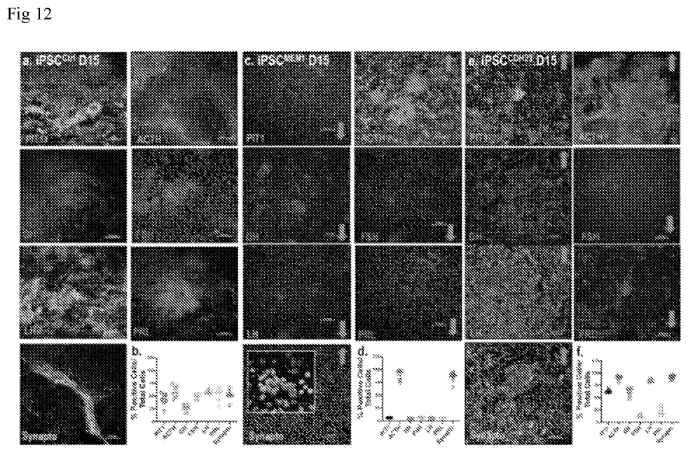

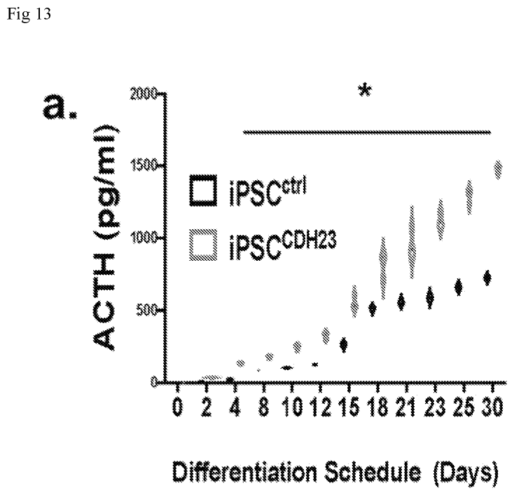

Pituitary organoids were then developed from iPSCs generated from the PBMCs of patients with CD (iPSC^{CDH23} and iPSC^{MEN1}) and a healthy individual (iPSC^{ctrl}). Expression of PIT1 (pituitary-specific positive transcription factor 1), ACTH (adrenocorticotropic hormone), GH (growth hormone), FSH (follicle-stimulating hormone), LH (luteinizing hormone), PRL (prolactin) and synaptophysin (synaptophysin) with co-stain Hoechst (nuclei, blue) was measured by immunofluorescence using chamber slides collected at 15 of the differentiation schedules (FIG. 12). While pituitary tissue that was differentiated from iPSCCtrl expressed all major hormone-producing cell lineages (FIG. 12a), there was a significant increase in the expression of ACTH and synaptophysin with a concomitant loss of PIT1, GH, FSH, LH and PRL in iPSCs^{MEN1} (FIGS. 12b, 12c). Interestingly iPSCCDH23 cultures exhibited significant increase in the expression of ACTH, GH, LH and synaptophysin with a concomitant loss of PIT1, FSH and PRL (FIGS. 12b, 12c). Immunofluorescence of iPSCs collected at day 4 of the differentiation schedule revealed no expression of PIT1, ACTH, GH, FSH, LH and PRL in (data not shown). Compared to control lines, iPSC lines expressing mutated CDH23 secreted significantly greater concentrations of ACTH earlier in the differentiation schedule (FIG. 13a). The upregulated expression of pituitary corticotroph adenoma-specific markers in iPSCCDH23 and iPSCMEN1 demonstrates that the iPSC-derived organoids represented the pathology of corticotroph adenomas in vitro.

### Example 8 ScRNA-Seq Reveals the Existence of Unique Proliferative Cell Populations in iPSC^{CDH23} Cultures when Compared to iPSCs^{ctrl}

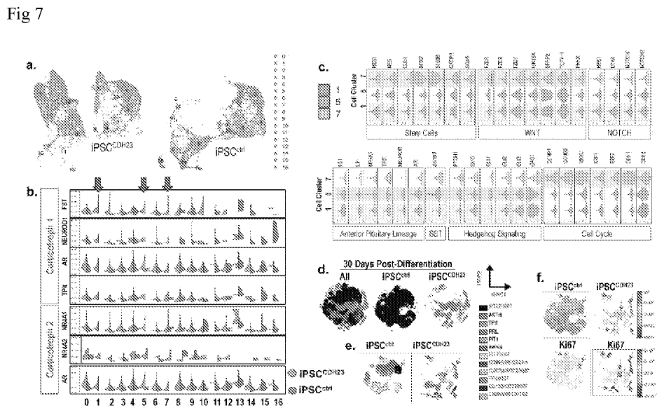

Using SEURAT to identify cell clusters and Uniform Manifold Approximation and Projection 9UMAP), clustering analysis identified 16 distinct cell populations/clusters consisting of known marker genes. Clusters 1, 5 and 7 of the iPSCs^{CDH23} were distinct from the iPSC^{ctrl} cultures (FIGS. 7a, 7b). Pituitary stem cells were characterized in iPSC^{ctrl} and iPSCCDH23 cultures (FIG. 7b). Clusters 1 and 5 expressed markers consistent with the corticotroph subtype cell lineage (FIG. 5c). Markers of dysregulated cell cycle and increased proliferation were identified in cell cluster 7 (FIG. 7c). Expression of the E2 factor (E2F) family of transcription factors, that are downstream effectors of the retinoblastoma (RB) protein pathway and play a crucial role in cell division control, were identified in distinct cell cluster 7 identified within the iPSC^{CDH23} cultures (FIG. 7c). Stem cell markers were also upregulated in cell cluster 7 identified within the iPSC^{CDH23} cultures (FIG. 7c). Using Cytobank software to analyze organoids collected 30 days post-differentiation, cells were gated on live CK20 positive singlets and 9000 events per sample were analyzed by the viSNE algorithm. ViSNE plots are shown in two dimensions with axes identified by tSNE-1 and tSNE-2 and each dot representing a single cell positioned in the multidimensional space (FIG. 7d). Individual flow cytometry standard files were concatenated into single flow cytometry standard files from which 12 spatially distinct populations were identified (FIG. 7e). Overlaying cell populations identified by traditional gating strategies on to viSNE plots identified unique cell populations within the iPSC^{CDH23} cultures (FIG. 7e). There were distinct cell populations between the iPSC^{ctrl} and iPSC^{CDH23} organoids in addition to expression of hormone and cell lineage markers such as ACTH, TPit, PRL and PIT1 (FIG. 7e). The cell populations that exhibited high expression of Ki67 within the iPSC^{ctrl} organoid cultures included SOX2+, and PIT1+ populations (FIG. 7f). The highly proliferating cell populations within the iPSC^{CDH23} organoid cultures included those that expressed CD90+/VIM+/CXCR4+ (mesenchymal stem cells), CXCR4+/SOX2+ (stem cells), TPit+ (corticotroph cell lineage), CD133+/CD31+ (endothelial progenitor cells), and CK20+/VIM+/CXCR4+ (hybrid epithelial-mesenchymal stem cells) (FIG. 7f). Overall, the iPSC^{CDH23} organoids were significantly more proliferative compared to the iPSC^{ctrl} cultures (FIG. 7f). Immunofluorescence staining of iPSC^{CDH23} organoids revealed increased mRNA expression of TPit and POMC that correlated with increased ACTH protein, compared to iPSCs^{ctrl} (FIG. 12). As shown in FIGS. 12b and 12c, iPSC^{CDH23} cultures also exhibited a significant increase in the expression of GH and LH (FIGS. 12b, 12c).

Collectively, FIG. 7 demonstrates that the development of pituitary organoids generated from iPSCs of CD patients may reveal the existence of cell populations that potentially contribute to the support of adenoma growth and progression, and an expansion of stem and progenitor cells that may be the targets for tumor recurrence.

### Example 9 IPSC-Derived Pituitary Organoids Expressing Somatic Mutations Reveal Corticotroph PitNET Pathology Consistent with CD

Extensive research has revealed the role of somatic mutations in the development of CD adenomas. To study these mechanisms, we generated human pituitary organoids developed from iPSCs. With reference to a published protocol using embryonic stem cells together with the knowledge of the growth factor and genetic regulation of pituitary gland development, we optimized our approach for the development of pituitary organoids from human blood-derived iPSCs for gene editing. The iPSC lines were generated to express known somatic mutations in iPSC^{USP48} (Gene ID: 84196; M415I (G1245A) and M415V (A1243G)), or iPSC^{USP8} [25, 34, 37-42]. USP48 mutations included either M415V or M415I, designating iPSC^{USP48MV} and iPSC^{USP48MI} respectively. Successful gene editing was validated using primer specific visualization of a change in the restriction pattern at the site of interest. Brightfield images showed morphological variations between the control (iPSC^{ctrl}) and mutant iPSCs as early as day 4 and at day 15. Morphological changes and proliferation were obvious when iPSCs were embedded in Matrigel™ to generate organoids.

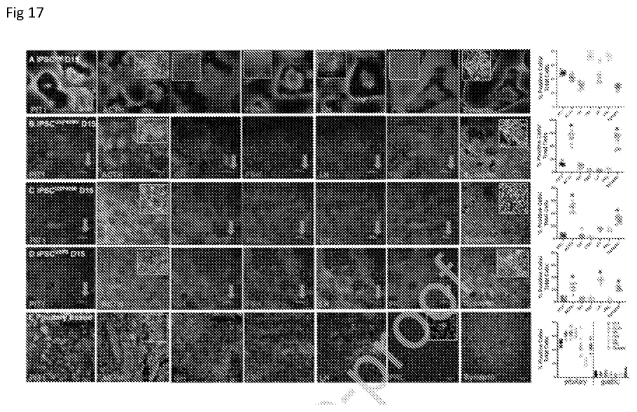

Expression of PIT1 (pituitary-specific positive transcription factor 1), ACTH (adrenocorticotropic hormone), GH (growth hormone), FSH (follicle-stimulating hormone), LH (luteinizing hormone), PRL (prolactin) and synaptophysin (synapto) with co-stain Hoechst (nuclei, blue) was measured by immunofluorescence using chamber slides collected at days 4 and 15 (D15, FIG. 17) of the differentiation schedules. While pituitary tissue that was differentiated from control iPSCs (iPSC^{ctrl}) expressed all major hormone-producing cell lineages (FIG. 17A), there was a significant increase in the expression of ACTH and synaptophysin with a concomitant loss of PIT1, GH, FSH, LH and PRL in iPCSs expressing mutated USP48 and USP8 (FIGS. 17B, 17C, 17D). Positive immunofluorescence staining of pituitary tumor tissue was used as a positive control and is shown in FIG. 17E. Gastric tissue was used as a negative control. Immunofluorescence of iPSCs collected at day 4 of the differentiation schedule revealed no expression of PIT1, ACTH, GH, FSH, LH and PRL in iPSC^{ctrl}. Unexpectedly, ACTH expression was increased in iPSC^{USP48} at day 4.

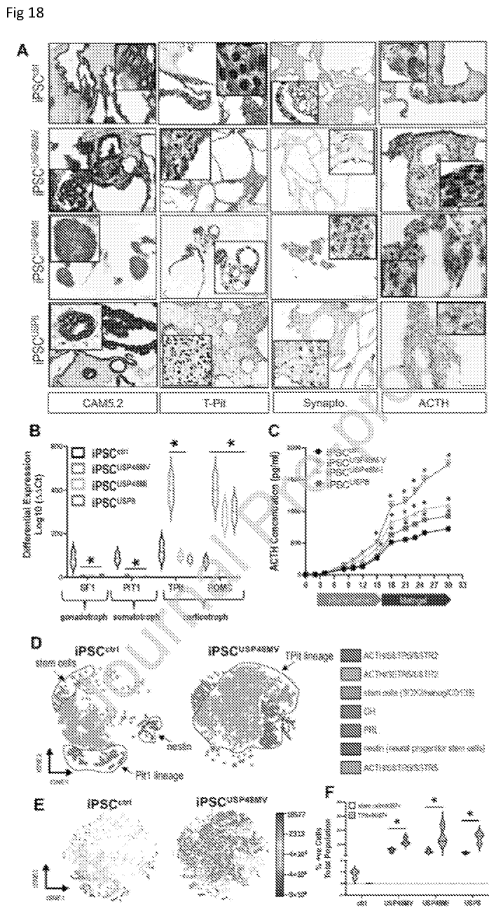

Immunohistochemistry of formalin fixed and paraffin embedded iPSC^{ctrl}, iPSCUSP48, and iPSC^{USP8} expressed CAM5.2, T-PIT, synaptophysin and ACTH (FIG. 18A, higher magnification of image is shown in the inset). The iPSC^{ctrl} line expressed transcription factors TPit (TBX19), PIT1 (POU1F1) and SF1 that corresponded to the differentiation of corticotrophs, somatotrophs and gonadotrophs as documented by positive immunofluorescence staining (FIG. 18B). The iPSC lines expressing mutations in USP48MV exhibited significantly elevated transcription factor T-Pit consistent with the skewed differentiation in the corticotroph cell lineage (FIG. 18B). Transcription factors PIT1 (somatotrophs) and SF1 (gonadotroph) were significantly decreased in the cultures of iPSC lines expressing USP48MV compared to iPSC^{ctrl} (FIG. 18B). Culture conditioned media was collected during the differentiation schedule of the iPSCs and analyzed for ACTH secretion by ELISA (FIG. 18C). Compared to control lines, iPSC lines expressing mutated USP8 and USP48 secreted significantly greater concentrations of ACTH earlier in the differentiation schedule (FIG. 18C).

The expression pattern of corticotroph PitNET specific markers was analyzed using Cytek™ Aurora spectral flow cytometry (FIG. 18D). The location of cells that were found in each cluster based on the highly expressed antigens are shown in the representative t-SNE maps (FIG. 18D). Compared to iPSC^{ctrl} organoids, iPSC^{USP48} and iPSC^{USP8} contained higher numbers of cells expressing T-Pit cell lineage and ACTH/SSTR2/SSTR5 expressing cells (representative t-SNE map of iPSC^{USP48MV} shown in FIG. 18D). In addition, iPSC^{ctrl} organoids expressed a higher percentage of Pit1 cell lineage, GH and PRL positive cells (FIG. 18D). Within the iPSC^{USP48} and iPSC^{USP8} cultures, stem cells expressing SOX2+/nanog+/CD133+ were significantly more abundant compared to the iPSC^{ctrl} organoids (FIG. 18D). Heatmap dot plots obtained after tSNE analysis showed that both the stem cell and TPit populations within the iPSC^{USP48} and iPSC^{USP8} organoid cultures expressed higher proliferating cells as measured by Ki67 (FIGS. 18E, 18F).

Differentiated iPSCs were then collected at D15 of the culture schedule and embedded into Matrigel™. Proliferation was quantified by EdU uptake of the cells within the pituitary organoids. Compared to control pituitary organoids iPSC^{ctrl}, organoids expressing mutations in USP8 (iPSCUSP8), and USP48 (iPSC^{USP48MV} and iPSCUSP48MI) expressed significantly elevated Edu+ proliferating cells. Collectively, these data show that iPSCs genetically engineered to express somatic mutations relevant to the development of CD exhibit corticotroph PitNET pathology and function in vitro.

### Example 10 Genetically Engineered iPSCs Expressing Somatic Mutations Relevant to CD Identify Differential Effects of Relacorilant and Mifepristone on SSTR Expression, and Tumor Cell Proliferation and Apoptosis

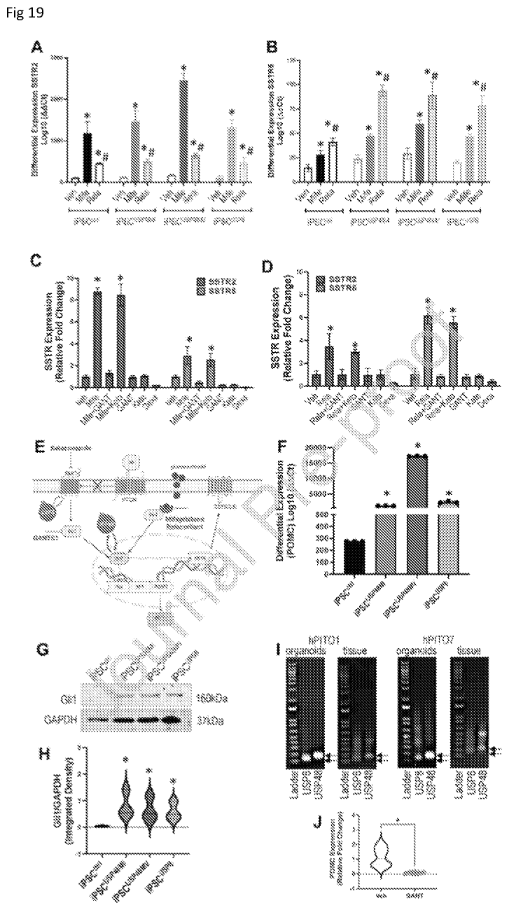

The treatment of iPSC organoids with Mifepristone or relacorilant resulted in a significant induction in the expression of SSTR2 and 5 (FIGS. 19A, 19B). Mifepristone led to a significantly greater induction in SSTR2 expression when compared to relacorilant (FIG. 19A). In contrast, relacorilant induced greater SSTR5 expression (FIG. 19B).

To identify the role Hedgehog signaling as a mediator of Mifepristone or relacorilant regulated SSTR expression, iPSC^{ctrl} organoids were used in combination with inhibitors GANT61 (GLI inhibitor) and ketoconazole (an SMO inhibitor that also inhibits cortisol synthesis) (FIGS. 19C, D). Mifepristone significantly induced the differential expression of both SSTRs 2 and 5, and this increase was reduced with GANT61 pretreatment of the organoid cultures (FIG. 19C). In contrast to the inhibitory effect of GANT61 on Mifepristone-induced SSTR expression, ketoconazole pretreatment had no effect on this induction (FIG. 19C). GANT61 and ketoconazole alone had no effect on the differential expression of SSTRs 2 and 5, while dexamethasone, a known GR agonist, significantly decreased expression of these receptors (FIG. 19C). Similar responses were observed with relacorilant with or without GANT61 and ketoconazole (FIG. 19D). These results suggest that Mifepristone and relacorilant induce SSTR2 and SSTR5 via an SMO-independent non-canonical Hh signaling pathway.

Organoids expressing both the USP8 and USP48 mutations exhibited characteristics consistent of corticotroph subtype PitNETs (FIGS. 17-19). Based on initial evidence in the literature, USP48 and USP8 mutations in PitNETs are believed to enhance corticotropin releasing hormone (CRH)-induced production coherent with the Hh signaling pathway (FIG. 19E). Hh ligand, Sonic Hedgehog (Shh), binds to Patched (Ptch1) that relieves suppression of Smoothened (Smo) and subsequently Gli1 activation (FIG. 19E). Crosstalk between Shh and CRH at the Gli1 level stimulates POMC transcription and ACTH secretion. Our data is consistent with the hypothesis that mutations in USP48 lead to increased levels of Gli1 and enhancing POMC transcription via an unknown a mechanism yet to be revealed (FIG. 19E). USP8 was shown to promote SMO activity that may subsequently also lead to POMC transcription and ACTH secretion (FIG. 19E) [47]. Our proposed mechanism is supported by significantly greater differential expression of POMC (FIG. 19F) in the iPSC^{USP48} and iPSC^{USP8} organoids when compared to the control cultures. In addition, GLI1 protein expression was significantly increased in the iPSC^{USP48} and iPSC^{USP8} organoids compared to the iPSC^{ctrl} cultures (FIGS. 19G, 19H). Mutations in USP8 and USP48 detected in hPITO cultures (hPITO1 and 7) were also expressed in the patient's matched PitNET tissue (FIG. 191). Gene ChIP assay revealed that the transcriptional regulation of POMC by Gli1 was blocked by GANT61 treatment of cultures (FIG. 19J).

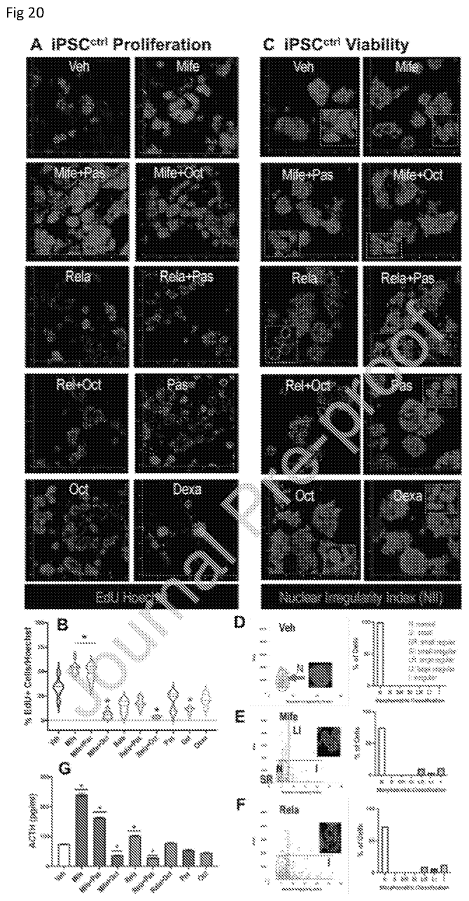

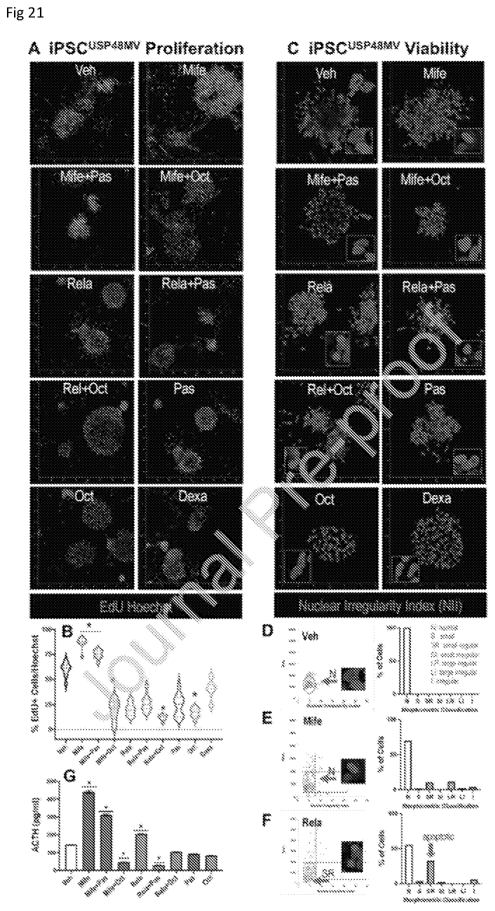

Analysis of proliferation using EdU uptake in iPSC^{ctrl} and iPSC^{USP48MV} organoid cultures showed that Mife induced a significant increase in cell proliferation (FIGS. 20A, 20B and FIGS. 21A, 21B). While the proliferative response to Mife was inhibited by pretreatment with Oct, Pas had no effect (FIGS. 20A, 20B and FIGS. 21A, 21B). While a similar response was observed in Rela treated cultures with pretreatment with Pas or Oct, Rela alone did not induce organoid proliferation (FIGS. 20A, 20B and FIGS. 21A, 21B). The nuclear irregularity index (NII) was measured based on the quantification of the morphometric changes in the nuclei in response to the treatment groups in the iPSC^{ctrl} (FIGS. 20C-F) and iPSC^{USP48MV} organoids (FIGS. 21C-F). While Rela induced a significant expression pattern of nuclear morphology consistent with increased apoptotic cells in the iPSC^{USP48MV} cultures (FIGS. 21C-F), this was not observed in the iPSC^{ctrl} organoids (FIGS. 20C-F). The magnitude of the apoptotic response to Rela was not observed with Mife in either the iPSC^{ctrl} (FIGS. 20C-F) or iPSC^{USP48MV} (FIGS. 21C-F) organoids.

Within the iPSC^{ctrl} and iPSC^{USP48MV} I organoid cultures, the magnitude of ACTH secretion induced by Mifepristone was significantly greater than the effect of relacorilant (FIGS. 20G and 21G). Pas and Oct significantly reduced ACTH secretion in response to Mifepristone, although the inhibition by Octreotide was greater that Pasireotide (FIG. 20G and FIG. 21G). Pasireotide reduced hormone secretion in combination with relacorilant at a greater magnitude compared to that of Octreotide plus relacorilant in iPSC^{ctrl} and iPSC^{USP48MV} organoid cultures (FIG. 20G and FIG. 21G).

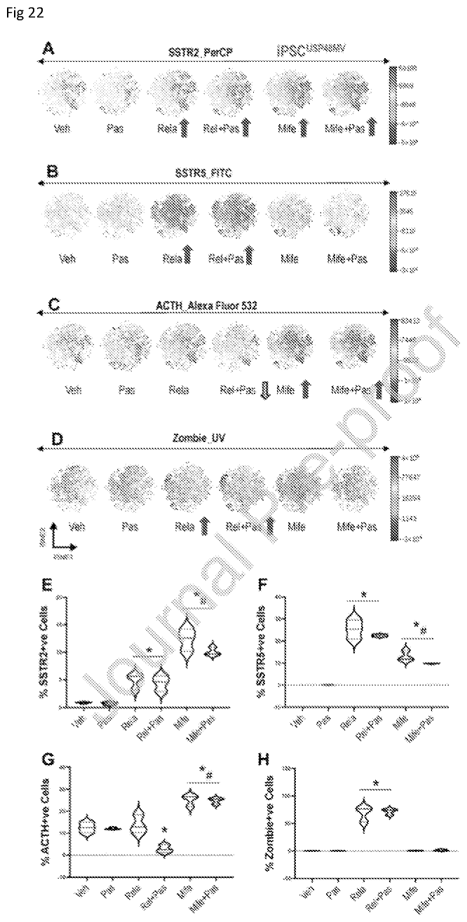

Heatmap dot plots obtained after tSNE analysis of iPSC^{USP48MV} organoids showed the relative expression of the SSTR2, SSTR5, ACTH and zombie positive cells in the different phenotypic clusters in response to Rela and Mife (FIG. 22). Quantification based on the gated identified cell clusters revealed that both Rela and Mife induced a significant increase in percentage of SSTR2 (FIGS. 22A, 22E) and SSTR5 (FIGS. 22B, 22F) positive cells. However, the magnitude of Mife induction of SSTR2 was significantly greater than that of Rela (FIGS. 22A, 22E). Similarly, the magnitude of Rela induction of SSTR5 was significantly greater than that of Mife (FIGS. 22B, 22F). In contrast to Rela, Mife led to a significant induction in the number of ACTH positive cells (FIGS. 22C, 22G). Rela did not significantly increase ACTH, and Pas+Rela significantly reduced ACTH expression in cultures (FIGS. 22C, 22G). Rela, or Rela plus Pas, clearly induced iPSC^{USP48MV} cell death as measured by the significant increase in Zombie positive cells that co-expressed ACTH and stem cell markers, a response not observed with Mife (FIGS. 22D, 22H).

To assess GR modulation, the GR target gene FKBP5 was measured in the PitNET organoids. While Mifepristone and relacorilant significantly reduced the differential expression of FKBP5, dexamethasone caused a significant induction in gene expression. Compared to Mifepristone, the magnitude of FKBP5 inhibition was significantly greater in response to relacorilant. This confirmed GR modulation in the organoid cultures in response to Mifepristone and relacorilant.

Collectively, these data demonstrate that, compared to relacorilant, Mifepristone preferentially induced the expression of SSTR2, ACTH secretion and PitNET organoid proliferation. In contrast, relacorilant predominantly induced SSTR5 expression and PitNET organoid apoptosis.

### Example 11 Relacorilant Induces Tumor Cell Death and Reduces ACTH in Combination with Pasireotide in Organoids Generated from CD Patient PitNETs

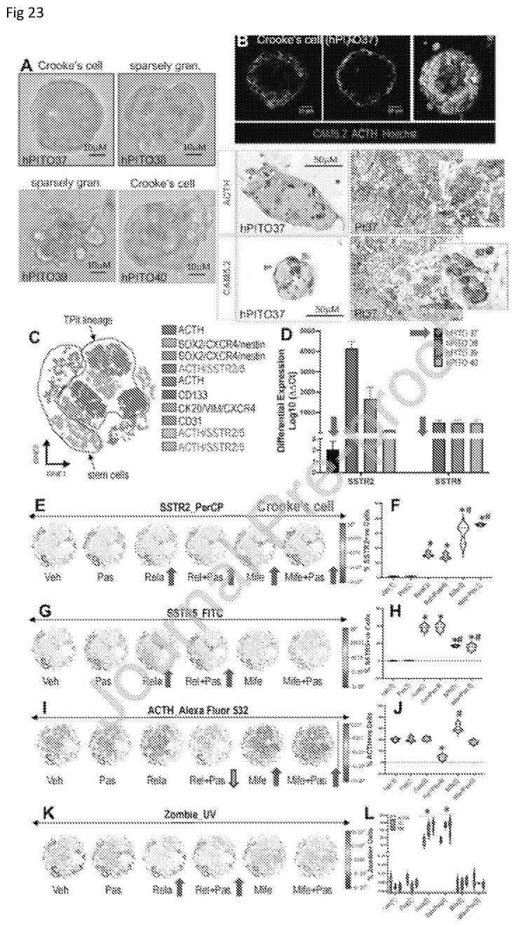

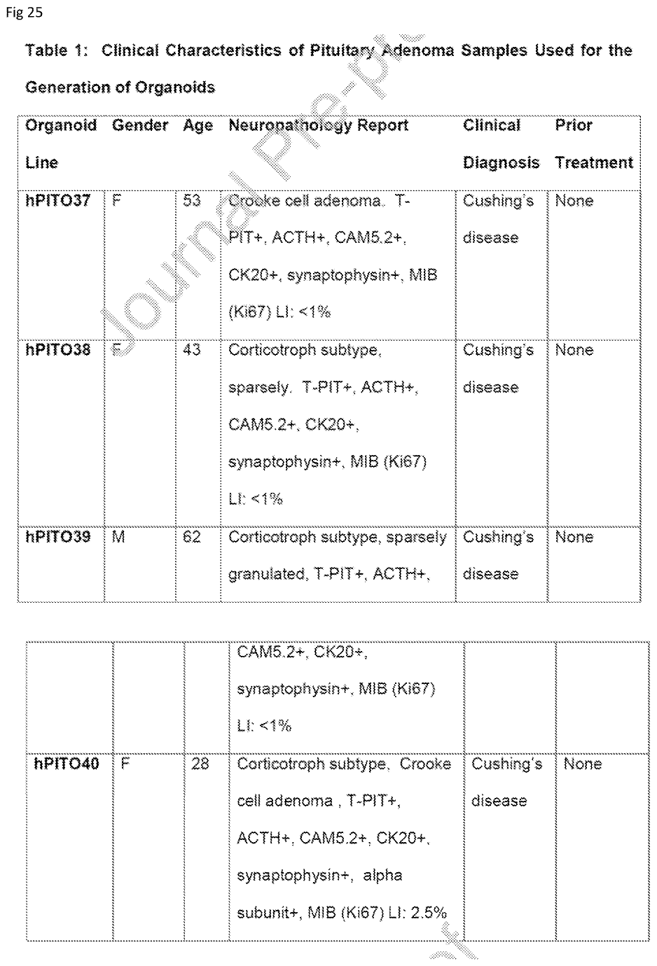

As part of a previous study, we generated an initial biobank of organoids generated from CD patient PitNETs. Human PitNET tissue was harvested during endoscopic transsphenoidal pituitary surgery from 40 patients to generate organoids. These cultures are referred to as human PitNET derived organoids (hPITOs). In FIG. 25, Table 1 summarizes the neuropathology reports and clinical diagnosis from cases used to generate hPITOs 37, 38, 39, 40 reported in the current study. Bright-field microscopy images of hPITOs that were generated from corticotroph PitNETs from patients diagnosed with CD revealed morphological diversity, including size, density, and regularity, among the organoid lines between individual patients and between Crooke's cell adenoma and sparsely granulated subtypes (FIG. 23A). Immunofluorescence confocal microscopy showed the expression of ACTH and CAM5.2 (cytokeratin 8/18) pituitary corticotroph markers (representative image of Crooke's cell hPITO37 shown in FIG. 23B). Consistent with the immunofluorescence, immunohistochemistry of FFPE sections of hPTO37 organoid line and the patient's matched PitNET showed positive staining for both ACTH and CAM5.2 (FIG. 23B). These data were consistent with the neuropathology report (FIG. 25).

To further test the similarity in cell lineages identified between the organoid line and the patient's tumor, we compared the immunohistochemistry from the neuropathology reports of the patients detailed in Table 1 in FIG. 25, to the expression pattern of pituitary tumor specific markers that were measured using Cytek™ Aurora spectral flow cytometry (FIG. 23C). Flow cytometric analysis using Cytobank revealed that hPITOs derived from patients with CD expressed increased stem and progenitor cell markers including CXCR4, SOX2, and CD133 (FIG. 23C). Using a gating strategy for the TPit positive cells, we identified ACTH positive cell populations co-expressing SSTR2 and SSTR5 (FIG. 23C). Diversity in the differential expression of SSTR2 and SSTR5 among the different hPITO lines was observed when SSTR2 and SSTR5 gene expression was measured (FIG. 23D).

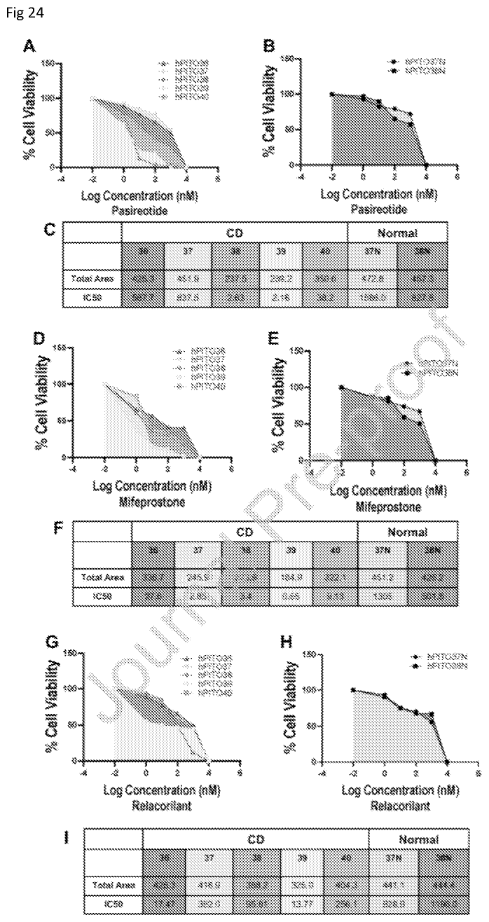

The calculation of the total area under the curve (FIG. 24) based on the dose response curve for each organoid line showed divergent responses to Mifepristone, Pasireotide and relacorilant in individual cultures (FIGS. 24A-I). While differential IC50 values were observed among the individual PitNET organoid lines (FIGS. 24A, 24D, 24G), this divergence in sensitivity was not reported in cultures prepared from the matched adjacent normal pituitary tissues (FIGS. 24B, 24E, 24H). Organoid line hPITO37 (Crooke's cell adenoma subtype) expressed significantly lower SSTR2 and SSTR5 expression levels compared to the other cultures (FIG. 23D) and was insensitive to Pas (FIG. 24). Therefore, we used this hPITO line to identify the differential effects of Mife and Rela on receptor expression and cell viability. Heatmap dot plots obtained after tSNE analysis of hPITO cultures showed the relative expression of the SSTR2, SSTR5, ACTH and zombie positive cells in the different phenotypic clusters in response to Rela and Mife (FIGS. 23E-L). Quantification based on the gated identified cell clusters revealed that both Rela and Mife induced a significant increase in percentage of SSTR2 and SSTR5 positive cells (FIGS. 23E-H). However, the magnitude of Mife induction of SSTR2 was significantly greater than that of Rela (FIGS. 23E, 23F). While both Rela and Mife induced a significant increase in percentage SSTR5 positive cells, the magnitude of Rela induction of SSTR5 was significantly greater than that of Mife (FIGS. 23G, 23H). In contrast, Mife significantly increased the number of ACTH expressing cells in culture, a response that was not inhibited by Pas pretreatment (FIGS. 231, 23J). Rela clearly induced hPITO cell death as measured by the significant increase in Zombie positive cells in response to Rela or Rela plus Pas, a response not observed with Mife (FIGS. 23K, 23L). In contrast to Mife, Rela also induced significant cell death in cell populations expressing stem cell markers SOX2, CXCR4 and nestin, and an epithelial/mesenchymal hybrid cell population that co-expressed CK20, vimentin and CXCR4 (FIGS. 23K, 23L). Therefore, Rela sensitizes CD PitNET tissue derived organoids to Pasireotide and induces cell death in several different morphologically diverse cell populations.

Lack of effective medical therapies targeted directly to the corticotroph PitNETs is potentially attributed to the lack of human patient-relevant model systems that recapitulate the cellular complexity of the tumor. Our studies contribute to overcoming this challenge by generating a PitNET organoid model system that is applied to identifying the mechanisms of action of Mifepristone and relacorilant (alone and in combination with other medical therapies for Cushing's disease). As part of our studies, PitNET organoids were generated from: 1) CRISPR-Cas9 gene editing of patient iPSCs, and 2) CD patient corticotroph PitNETs (hPITOs). Using these advanced in vitro culture systems, we demonstrated that SSTR2 and SSTR5 are targets of Hedgehog transcription effector Gli1 and this response is attenuated by activation of the GR pathway. In these experiments, we have shown that Mifepristone and relacorilant exerted differential effects on the induction of SSTR2 and SSTR5 expression, ACTH secretion and PitNET organoid proliferation and apoptosis.

While both Mifepristone and relacorilant significantly induced the expression of SSTR2 and SSTR5, the magnitude of induction of these receptors was different. While Mifepristone predominantly induced SSTR2 expression, relacorilant predominantly induced SSTR5. The SSTRs expressed on the surface of corticotroph PitNETs have often been targeted for the treatment of CD. Multiple studies suggest that SSTR5 is consistently overexpressed in corticotroph PitNETs, however, treatments with somatostatin analogues (Octreotide and Pasireotide) are designed without considering variations in receptor subtype expression among individual patients with CD. We clearly show the presence of variation in the SSTR2 and SSTR5 expression amongst individual patients with CD, and, importantly, SSTR5 was significantly upregulated by relacorilant and subsequently sensitized organoid tumor cells to suppression of ACTH by Pasireotide. Our findings are consistent with the knowledge that Pasireotide has high-affinity binding to SSTR5 [20]. In fact, Pasireotide monotherapy normalizes cortisol in up to 42% of patients with CD but assumes that all patients express this receptor subtype. Combination treatment using Pasireotide administered together with cabergoline and ketoconazole may increase the efficacy of the somatostatin analogue, but there is no clear consideration for changes in SSTRs during treatment. Therefore, our findings suggest that treating patients with CD with relacorilant may increase the efficacy of Pasireotide (or, potentially, endogenous somatostatin) suppression of ACTH secretion from corticotroph PitNETs.

In our studies we showed that Hedgehog (Hh) signaling mediates SSTR2 and 5 expression that is induced by Mifepristone and relacorilant through a non-canonical Hh signaling pathway. In the iPSC organoids, GLI inhibitor GANT61 inhibited both Mifepristone and relacorilant stimulated SSTR2 and SSTR5. The SMO inhibitor ketoconazole (which also inhibits cortisol synthesis) failed to block the induction of both SSTR2 and SSTR5 by both compounds. Upon ligand binding to the transmembrane receptor Patched-1 (PTCH1), the repression of PTCH1 on transmembrane transducer SMO is released. De-repressed SMO triggers the activation of GLI zinc finger transcription factors GLI1, GLI2 and GLI3. Activated GLI1 protein translocates into the nucleus, where several target genes are regulated [49]. Our studies suggest that within the corticotroph PitNETs, SSTR2 and SSTR5 are transcriptional targets of GLI. The failure of the SMO inhibitor ketoconazole to inhibit Mifepristone and relacorilant induction of SSTR expression suggests that the signaling pathway is non-canonical. Non-canonical Hh signaling pathways have been classified as 1) Type I pathways involving signaling through PTCH1 independent of SMO, 2) Type II pathways involving signaling through SMO independent of GLI, and 3) SMO-independent activation of GLI such as that observed in the current study. In support of our findings, crosstalk between GLI and GR (NR3C1) signaling has been reported in T cell acute lymphoblastic leukemia. In addition, studies in the adult stomach demonstrated that the Hh pathway is critical for the regulation of somatostatin and SSTR signaling. While Hh signaling is known to be essential during the embryonic development of the pituitary and in the adult gland [54], this is the first report demonstrating that Hh signaling via Gli1 regulates the expression of SSTR2 and SSTR5 in PitNET organoids.

The expression of USP8 and USP48 mutations increased the differentiation of the iPSC generated organoids consistent with a corticotroph subtype PitNET phenotype. Our data and evidence in the literature shows that corticotroph subtype PitNETs expressing USP8 and USP48 mutations target members of the Hh signaling pathway including SMO and Gli1 respectively. Published studies show that Hh ligand Shh and the activation of the signaling pathway is a key regulator of the stem/progenitor cell proliferation in both normal pituitary and PitNETs that may promote the development of corticotroph subtype PitNETs. Thus, a plausible explanation for the tumorigenesis of corticotroph subtype PitNETs is that Hh signaling drives the increased proliferation and POMC expression of stem/progenitor cells.

In contrast to Mifepristone, relacorilant induced PitNET cell death in treated organoid cultures without significant stimulation of ACTH secretion. In concurrence with the organoid in vitro studies, tumor regression in two patients with macroadenomas treated with relacorilant for three months have been reported in an initial study based on standard of care imaging of the pituitary gland. This unexpected finding is further being investigated in an ongoing Phase 3 study of relacorilant in patients with Cushing's syndrome (GRACE Study, NCT03697109). Such macroadenoma regressions have not been observed with Mifepristone. Based on the negative feedback mechanism regulating the hypothalamic-pituitary-adrenal (HPA) axis, we propose the following mechanisms to explain the differential effects between Mifepristone and relacorilant. First, Mifepristone predominantly induced SSTR2 expression. However, unlike relacorilant, Mifepristone treatment significantly increased ACTH secretion from PitNET both in vivo and in vitro. Our organoid experiments are supported by patient data showing that Mifepristone increases ACTH, with a concomitant increase in cortisol secretion without tumor growth. In contrast, relacorilant predominantly induced SSTR5 expression without an increase in ACTH secretion and tumor cell death. A plausible explanation for PitNET regression in response to relacorilant treatment may be the known heterodimerization between SSTR2 and SSTR5 that occurs following the selective activation of SSTR2 but not human SSTR5 or concurrent stimulation. Heterodimerization between SSTR2 and SSTR5 leads to an increased recycling rate and a greater propensity of SSTR2 to induce tumor growth inhibition. Thus, in vivo, the significant effect of Mifepristone on SSTR2 and increased receptor signaling offsets the proliferative effects of this antagonist and may explain the lack of tumor growth in treated patients. Relacorilant may also induce PitNET apoptosis independently of SSTR activation. In support of this notion, studies in several cancers that are driven by the dysregulation of these receptors, including leukemia, breast, prostate, lung and ovarian cancers, show promise of targeting the GR as a strategy for combination treatment. In addition, relacorilant induces apoptosis in vitro using pancreatic and ovarian cancer cell lines [63]. The pro-apoptotic effects of GR modulators are tissue specific and may be a result of targeting the apoptotic/cycle signaling pathways and genes such as Bcl2 [64]. The mechanism of action of relacorilant on PitNET cell apoptosis requires further investigation. Collectively, these studies support broader investigation of relacorilant alone or in combination with somatostatin analogs as pre- or post-operative medical treatment in patients with CD.

While many investigators have proposed using organoids in personalized medicine for the targeted treatment of cancers, this study is the first to execute this approach for the potential treatment of corticotroph type PitNETs associated with the development of CD. Existing medical therapy for CD remains suboptimal with negative impact on health and quality of life, including the considerable risk of therapy resistance and tumor recurrence. Our data demonstrate that organoids derived from corticotroph type PitNETs consist of differentiated cell lineages, stem/progenitor cells, and stromal cells that replicate much of the patient's own tumor pathology and function clearly documented by extensive high-throughput flow cytometry. Previously published in vitro experiments in the field of CD research were performed using pituitary cell lines or spheroids, aggregates and tumoroids that do not replicate the primary pituitary tumor microenvironment due to cell transformation and/or unphysiological 2D culture conditions. Pituitary research has largely been conducted using cell culture techniques using rat (GH3) or mouse (AtT20) pituitary-like cell lines lacking a multicellular identify reflective of the PitNET. Recent advances have led to the development of pituitary tissue generated organoids, but these are limited to the use of transgenic mouse models as the source. Nys et al. reported the generation of human pituitary tumor organoids from a single stem cell as claim of true organoids due to the clonality. Unlike our studies, the multicellular complexity was not validated by the protein expression or hormone secretion from pituitary cell lineages in these cultures. It is fundamental to note that according to the National Cancer Institute (NCI, NIH), an ‘organoid’ is defined as “A tiny, 3-dimensional mass of tissue that is made by growing stem cells (cells from which other types of cells develop) in the laboratory.” Our cultures begin with both single and 3-4 cell clusters isolated from the native CD patient PitNET tissue that harbor the stem cells and begin a process of ‘budding’, growth and differentiation and the comprehensive spectral flow cytometry analysis documenting functional cell lineages. Our PitNET organoids are consistent with gastrointestinal tissue derived organoids including that begin from cell clusters, crypts or glands. Importantly, the videos show a different process than what would be an expected observation for the formation of a tumoroid which is the migration and adhesion of initially separate cells to form an aggregate. There are the reports of mouse nonadherent spheres with stem/progenitor characteristics, and human embryonic stem cell generated spheroids or patient derived tumoroids that also lack a multicellular identify and consist of poorly differentiated cells. Therefore, the findings reported here are significant because we offer an advanced in vitro approach that reveals cell populations expressing stem cell markers that potentially contribute to the support of tumor growth and targeted to prevent tumor recurrence. The ability of relacorilant to induce PitNET cell death in a multicellular culture system is the first step in the development of effective targeted therapies for patients with CD and potentially, other PitNET subtypes.

### REFERENCES

The references listed below are cited throughout the disclosure, some in the format of [number] (for example [1]) and are hereby incorporated by reference into this disclosure.
1. Cushing, H. Posterior Pituitary Activity from an Anatomical Standpoint. Am J Pathol 1933, 9, 539-548 519.
2. Cushing, H. The basophil adenomas of the pituitary body and their clinical manifestations (pituitary basophilism). 1932. Obes Res 1994, 2, 486-508, doi: 10.1002/j.1550-8528.1994.tb00097.x.
3. Ironside, N.; Chen, C. J.; Lee, C. C.; Trifiletti, D. M.; Vance, M. L.; Sheehan, J. P. Outcomes of Pituitary Radiation for Cushing's Disease. Endocrinol Metab Clin North Am 2018, 47, 349-365, doi: 10.1016/j.ecl.2018.01.002.
4. Loriaux, D. L. Diagnosis and Differential Diagnosis of Cushing's Syndrome. N Engl J Med 2017, 377, e3, doi: 10.1056/NEJMc1705984
5. Asa, S. L.; Mete, O.; Perry, A.; Osamura, R. Y. Overview of the 2022 WHO Classification of Pituitary Tumors. Endocr Pathol 2022, 33, 6-26, doi: 10.1007/s12022-022-09703-7.
6. Nishioka, H.; Yamada, S. Cushing's Disease. J Clin Med 2019, 8, doi: 10.3390/jcm8111951.
7. Feelders, R. A.; Hofland, L. J. Medical treatment of Cushing's disease. J Clin Endocrinol Metab 2013, 98, 425-438, doi: 10.1210/jc.2012-3126.
8. Limumpornpetch, P.; Morgan, A. W.; Tiganescu, A.; Baxter, P. D.; Nyawira Nyaga, V.; Pujades-Rodriguez, M.; Stewart, P. M. The Effect of Endogenous Cushing Syndrome on All-cause and Cause-specific Mortality. J Clin Endocrinol Metab 2022, 107, 2377-2388, doi: 10.1210/clinem/dgac265.
9. Ciric, I.; Zhao, J. C.; Du, H.; Findling, J. W.; Molitch, M. E.; Weiss, R. E.; Refetoff, S.; Kerr, W. D.; Meyer, J. Transsphenoidal surgery for Cushing disease: experience with 136 patients. Neurosurgery 2012, 70, 70-80; discussion 80-71, doi: 10.1227/NEU.0b013e31822dda2c.
10. Alexandraki, K. I.; Kaltsas, G. A.; Isidori, A. M.; Storr, H. L.; Afshar, F.; Sabin, I.; Akker, S. A.; Chew, S. L.; Drake, W. M.; Monson, J. P.; et al. Long-term remission and recurrence rates in Cushing's disease: predictive factors in a single-centre study. Eur J Endocrinol 2013, 168, 639-648, doi: 10.1530/EJE-12-0921.
11. Sonino, N.; Zielezny, M.; Fava, G. A.; Fallo, F.; Boscaro, M. Risk factors and long-term outcome in pituitary-dependent Cushing's disease. J Clin Endocrinol Metab 1996, 81, 2647-2652, doi: 10.1210/jcem.81.7.8675592.
12. van der Pas, R.; Feelders, R. A.; Gatto, F.; de Bruin, C.; Pereira, A. M.; van Koetsveld, P. M.; Sprij-Mooij, D. M.; Waaijers, A. M.; Dogan, F.; Schulz, S.; et al. Preoperative normalization of cortisol levels in Cushing's disease after medical treatment: consequences for somatostatin and dopamine receptor subtype expression and in vitro response to somatostatin analogs and dopamine agonists. J Clin Endocrinol Metab 2013, 98, E1880-1890, doi: 10.1210/jc.2013-1987.
13. Kondziolka, D. Cushing's disease and stereotactic radiosurgery. J Neurosurg 2013, 119, 1484-1485; discussion 1485, doi: 10.3171/2013.4.JNS13642.
14. Mehta, G. U.; Sheehan, J. P.; Vance, M. L. Effect of stereotactic radiosurgery before bilateral adrenalectomy for Cushing's disease on the incidence of Nelson's syndrome. J Neurosurg 2013, 119, 1493-1497, doi: 10.3171/2013.7.JNS13389.
15. Tritos, N. A. Adrenally Directed Medical Therapies for Cushing Syndrome. J Clin Endocrinol Metab 2021, 106, 16-25, doi: 10.1210/clinem/dgaa778.
16. Gheorghiu, M. L.; Negreanu, F.; Fleseriu, M. Updates in the Medical Treatment of Pituitary Adenomas. Horm Metab Res 2020, 52, 8-24, doi: 10.1055/a-1066-4592.
17. Kaiser, U. B. Cushing's disease: towards precision medicine. Cell Res 2015, 25, 649-650, doi: 10.1038/cr.2015.53.
18. Bissell, M. S. a. M. J. Organoids: A historical perspective of thinking in three dimensions. Journal of Cell Biology 2017
19. Danila, D. C.; Zhang, X.; Zhou, Y.; Dickersin, G. R.; Fletcher, J. A.; Hedley-Whyte, E. T.; Selig, M. K.; Johnson, S. R.; Klibanski, A. A human pituitary tumor-derived folliculostellate cell line. J Clin Endocrinol Metab 2000, 85, 1180-1187, doi: 10.1210/jcem.85.3.6424.
20. Bjoro, T.; Torjesen, P. A.; Ostberg, B. C.; Sand, O.; Iversen, J. G.; Gautvik, K. M.; Haug, E. Bombesin stimulates prolactin secretion from cultured rat pituitary tumour cells (GH4C1) via activation of phospholipase C. Regul Pept 1987, 19, 169-182, doi: 10.1016/0167-0115 (87) 90274-6.
21. Bjoro, T.; Sand, O.; Ostberg, B. C.; Gordeladze, J. O.; Torjesen, P.; Gautvik, K. M.; Haug, E. The mechanisms by which vasoactive intestinal peptide (VIP) and thyrotropin releasing hormone (TRH) stimulate prolactin release from pituitary cells. Biosci Rep 1990, 10, 189-199, doi: 10.1007/BF01116578.
22. Cox, B.; Laporte, E.; Vennekens, A.; Kobayashi, H.; Nys, C.; Van Zundert, I.; Uji, I. H.; Vercauteren Drubbel, A.; Beck, B.; Roose, H.; et al. Organoids from pituitary as a novel research model toward pituitary stem cell exploration. J Endocrinol 2019, 240, 287-308, doi: 10.1530/JOE-18-0462.
23. Vennekens, A.; Laporte, E.; Hermans, F.; Cox, B.; Modave, E.; Janiszewski, A.; Nys, C.; Kobayashi, H.; Malengier-Devlies, B.; Chappell, J.; et al. Interleukin-6 is an activator of pituitary stem cells upon local damage, a competence quenched in the aging gland. Proc Natl Acad Sci USA 2021, 118, doi: 10.1073/pnas.2100052118.
24. Zhang, D.; Hugo, W.; Redublo, P.; Miao, H.; Bergsneider, M.; Wang, M. B.; Kim, W.; Yong, W. H.; Heaney, A. P. A human ACTH-secreting corticotroph tumoroid model: Novel Human ACTH-Secreting Tumor Cell in vitro Model. EBioMedicine 2021, 66, 103294, doi: 10.1016/j.ebiom.2021.103294.
25. Tsukada, T.; Kouki, T.; Fujiwara, K.; Ramadhani, D.; Horiguchi, K.; Kikuchi, M.; Yashiro, T. Reassembly of anterior pituitary organization by hanging drop three-dimensional cell culture. Acta Histochem Cytochem 2013, 46, 121-127, doi: 10.1267/ahc.13015.
26. Narsinh, K. H.; Jia, F.; Robbins, R. C.; Kay, M. A.; Longaker, M. T.; Wu, J. C. Generation of adult human induced pluripotent stem cells using nonviral minicircle DNA vectors. Nat Protoc 2011, 6, 78-88, doi: 10.1038/nprot.2010.173.
27. Steele, N. G.; Chakrabarti, J.; Wang, J.; Biesiada, J.; Holokai, L.; Chang, J.; Nowacki, L. M.; Hawkins, J.; Mahe, M.; Sundaram, N.; et al. An Organoid-Based Preclinical Model of Human Gastric Cancer. Cell Mol Gastroenterol Hepatol 2019, 7, 161-184, doi: 10.1016/j.jcmgh.2018.09.008.
28. Bertaux-Skeirik, N.; Feng, R.; Schumacher, M. A.; Li, J.; Mahe, M. M.; Engevik, A. C.; Javier, J. E.; Peek, R. M. J.; Ottemann, K.; Orian-Rousseau, V.; et al. CD44 plays a functional role in Helicobacter pylori-induced epithelial cell proliferation. PLOS Pathog. 2015, 11 (2): e1004663. doi: 10.1371/journal.ppat. 1004663. eCollection 2015.
29. Feng, R.; Aihara, E.; Kenny, S.; Yang, L.; Li, J.; Varro, A.; Montrose, M. H.; Shroyer, N. F.; Wang, T. C.; Shivdasani, R. A.; et al. Indian Hedgehog mediates gastrin-induced proliferation in stomach of adult mice. Gastroenterology 2014, 147, 655-666 e659, doi: 10.1053/j.gastro.2014.05.006.
30. Filippi-Chiela, E. C.; Oliveira, M. M.; Jurkovski, B.; Callegari-Jacques, S. M.; da Silva, V. D.; Lenz, G. Nuclear morphometric analysis (NMA): screening of senescence, apoptosis and nuclear irregularities. PLOS One 2012, 7, e42522, doi: 10.1371/journal.pone.0042522.
31. Gagnon, R. C.; Peterson, J. J. Estimation of confidence intervals for area under the curve from destructively obtained pharmacokinetic data. J Pharmacokinet Biopharm 1998, 26, 87-102, doi: 10.1023/a: 1023228925137.
32. Livak, K.; Schmittgen, T. Analysis of relative gene expression data using real-time quantitative PCR and the 2 (−Delta Delta C (T)) Method. Methods 2001, 25, 402-408.
33. Hinojosa-Amaya, J. M.; Varlamov, E. V.; McCartney, S.; Fleseriu, M. Hypercortisolemia Recurrence in Cushing's Disease; a Diagnostic Challenge. Front Endocrinol (Lausanne) 2019, 10, 740, doi: 10.3389/fendo.2019.00740.
34. Patil, C. G.; Prevedello, D. M.; Lad, S. P.; Vance, M. L.; Thorner, M. O.; Katznelson, L.; Laws, E. R., Jr. Late recurrences of Cushing's disease after initial successful transsphenoidal surgery. J Clin Endocrinol Metab 2008, 93, 358-362, doi: 10.1210/jc.2007-2013.
35. Katznelson, L. Bilateral adrenalectomy for Cushing's disease. Pituitary 2015, 18, 269-273, doi: 10.1007/s11102-014-0633-2.
36. Reincke, M.; Sbiera, S.; Hayakawa, A.; Theodoropoulou, M.; Osswald, A.; Beuschlein, F.; Meitinger, T.; Mizuno-Yamasaki, E.; Kawaguchi, K.; Saeki, Y.; et al. Mutations in the deubiquitinase gene USP8 cause Cushing's disease. Nat Genet 2015, 47, 31-38, doi: 10.1038/ng.3166.
37. Chen, J.; Jian, X.; Deng, S.; Ma, Z.; Shou, X.; Shen, Y.; Zhang, Q.; Song, Z.; Li, Z.; Peng, H.; et al. Identification of recurrent USP48 and BRAF mutations in Cushing's disease. Nat Commun 2018, 9, 3171, doi: 10.1038/s41467-018-05275-5.
38. Zhang, Q.; Peng, C.; Song, J.; Zhang, Y.; Chen, J.; Song, Z.; Shou, X.; Ma, Z.; Peng, H.; Jian, X.; et al. Germline Mutations in CDH23, Encoding Cadherin-Related 23, Are Associated with Both Familial and Sporadic Pituitary Adenomas. Am J Hum Genet 2017, 100, 817-823, doi: 10.1016/j.ajhg.2017.03.011.
39. Ikeda, H.; Mitsuhashi, T.; Kubota, K.; Kuzuya, N.; Uchimura, H. Epidermal growth factor stimulates growth hormone secretion from superfused rat adenohypophyseal fragments. Endocrinology 1984, 115, 556-558.
40. Baek, N.; Seo, O. W.; Kim, M.; Hulme, J.; An, S. S. Monitoring the effects of doxorubicin on 3D-spheroid tumor cells in real-time. Onco Targets Ther 2016, 9, 7207-7218, doi: 10.2147/OTT.S112566.
41. Laporte, E.; Nys, C.; Vankelecom, H. Development of Organoids from Mouse Pituitary as In Vitro Model to Explore Pituitary Stem Cell Biology. J Vis Exp 2022, doi: 10.3791/63431.
42. Nys, C.; Lee, Y. L.; Roose, H.; Mertens, F.; De Pauw, E.; Kobayashi, H.; Sciot, R.; Bex, M.; Versyck, G.; De Vleeschouwer, S.; et al. Exploring stem cell biology in pituitary tumors and derived organoids. Endocr Relat Cancer 2022, 29, 427-450, doi: 10.1530/ERC-21-0374.
43. https://www.cancer.gov/publications/dictionaries/cancer-terms/def/organoid.
44. Mahe, M. M.; Aihara, E.; Schumacher, M. A.; Zavros, Y.; Montrose, M. H.; Helmrath, M. A.; Sato, T.; Shroyer, N. F. Establishment of Gastrointestinal Epithelial Organoids. Current Protocols in Mouse Biology 2013, 3, 217-240.
45. Schumacher, M. A.; Aihara, E.; Feng, R.; Engevik, A.; Shroyer, N. F.; Ottemann, K. M.; Worrell, R. T.; Montrose, M. H.; Shivdasani, R. A.; Zavros, Y. The use of murine-derived fundic organoids in studies of gastric physiology. J Physiol 2015, 593, 1809-1827, doi: 10.1113/jphysiol.2014.283028.
46. Holokai, L.; Chakrabarti, J.; Lundy, J.; Croagh, D.; Adhikary, P.; Richards, S. S.; Woodson, C.; Steele, N.; Kuester, R.; Scott, A.; et al. Murine- and Human-Derived Autologous Organoid/Immune Cell Co-Cultures as Pre-Clinical Models of Pancreatic Ductal Adenocarcinoma. Cancers (Basel) 2020, 12, doi: 10.3390/cancers12123816.
47. Boj, S. F.; Hwang, C. I.; Baker, L. A.; Chio, I I; Engle, D. D.; Corbo, V.; Jager, M.; Ponz-Sarvise, M.; Tiriac, H.; Spector, M. S.; et al. Organoid models of human and mouse ductal pancreatic cancer. Cell 2015, 160, 324-338, doi: 10.1016/j.cell.2014.12.021.
48. Tiriac, H.; Belleau, P.; Engle, D. D.; Plenker, D.; Deschenes, A.; Somerville, T. D. D.; Froeling, F. E. M.; Burkhart, R. A.; Denroche, R. E.; Jang, G. H.; et al. Organoid Profiling Identifies Common Responders to Chemotherapy in Pancreatic Cancer. Cancer Discov 2018, 8, 1112-1129, doi: 10.1158/2159-8290.CD-18-0349.
49. Driehuis, E.; van Hoeck, A.; Moore, K.; Kolders, S.; Francies, H. E.; Gulersonmez, M. C.; Stigter, E. C. A.; Burgering, B.; Geurts, V.; Gracanin, A.; et al. Pancreatic cancer organoids recapitulate disease and allow personalized drug screening. Proc Natl Acad Sci USA 2019, doi: 10.1073/pnas. 1911273116.
50. Jung, Y. H.; Choi, D. H.; Park, K.; Lee, S. B.; Kim, J.; Kim, H.; Jeong, H. W.; Yang, J. H.; Kim, J. A.; Chung, S.; et al. Drug screening by uniform patient derived colorectal cancer hydro-organoids. Biomaterials 2021, 276, 121004, doi: 10.1016/j.biomaterials.2021.121004.
51. Chen, D.; Tan, Y.; Li, Z.; Li, W.; Yu, L.; Chen, W.; Liu, Y.; Liu, L.; Guo, L.; Huang, W.; et al. Organoid Cultures Derived From Patients With Papillary Thyroid Cancer. J Clin Endocrinol Metab 2021, 106, 1410-1426, doi: 10.1210/clinem/dgab020.
52. Reincke, M.; Theodoropoulou, M. Genomics in Cushing's Disease: The Dawn of a New Era. J Clin Endocrinol Metab 2021, 106, e2455-e2456, doi: 10.1210/clinem/dgaa969.
53. Ma, Z. Y.; Song, Z. J.; Chen, J. H.; Wang, Y. F.; Li, S. Q.; Zhou, L. F.; Mao, Y.; Li, Y. M.; Hu, R. G.; Zhang, Z. Y.; et al. Recurrent gain-of-function USP8 mutations in Cushing's disease. Cell Res 2015, 25, 306-317, doi: 10.1038/cr.2015.20.
54. Melmed, S. Pathogenesis of pituitary tumors. Nat Rev Endocrinol 2011, 7, 257-266, doi: 10.1038/nrendo.2011.40.
55. Stratakis, C. A.; Tichomirowa, M. A.; Boikos, S.; Azevedo, M. F.; Lodish, M.; Martari, M.; Verma, S.; Daly, A. F.; Raygada, M.; Keil, M. F.; et al. The role of germline AIP, MEN1, PRKAR1A, CDKN1B and CDKN2C mutations in causing pituitary adenomas in a large cohort of children, adolescents, and patients with genetic syndromes. Clin Genet 2010, 78, 457-463, doi: 10.1111/j.1399-0004.2010.01406.x.
56. Mouchtouris, N.; Smit, R. D.; Piper, K.; Prashant, G.; Evans, J. J.; Karsy, M. A review of multiomics platforms in pituitary adenoma pathogenesis. Front Biosci (Landmark Ed) 2022, 27, 77, doi: 10.31083/j.fbl2703077.
57. Bolz, H.; von Brederlow, B.; Ramirez, A.; Bryda, E. C.; Kutsche, K.; Nothwang, H. G.; Seeliger, M.; del, C. S. C. M.; Vila, M. C.; Molina, O. P.; et al. Mutation of CDH23, encoding a new member of the cadherin gene family, causes Usher syndrome type 1D. Nat Genet 2001, 27, 108-112, doi: 10.1038/83667.
58. Zhang, S.; Cui, Y.; Ma, X.; Yong, J.; Yan, L.; Yang, M.; Ren, J.; Tang, F.; Wen, L.; Qiao, J. Single-cell transcriptomics identifies divergent developmental lineage trajectories during human pituitary development. Nat Commun 2020, 11, 5275, doi: 10.1038/s41467-020-19012-4.
59. Cheung, L. Y.; Davis, S. W.; Brinkmeier, M. L.; Camper, S. A.; Perez-Millan, M. I. Regulation of pituitary stem cells by epithelial to mesenchymal transition events and signaling pathways. Mol Cell Endocrinol 2017, 445, 14-26, doi: 10.1016/j.mce.2016.09.016.
60. Shintani, A.; Higuchi, M. Isolation of PRRX1-positive adult pituitary stem/progenitor cells from the marginal cell layer of the mouse anterior lobe. Stem Cell Res 2021, 52, 102223, doi: 10.1016/j.scr.2021.102223.
61. Yoshida, S.; Nishimura, N.; Ueharu, H.; Kanno, N.; Higuchi, M.; Horiguchi, K.; Kato, T.; Kato, Y. Isolation of adult pituitary stem/progenitor cell clusters located in the parenchyma of the rat anterior lobe. Stem Cell Res 2016, 17, 318-329, doi: 10.1016/j.scr.2016.08.016.
62. Laporte, E.; Vennekens, A.; Vankelecom, H. Pituitary Remodeling Throughout Life: Are Resident Stem Cells Involved? Front Endocrinol (Lausanne) 2020, 11, 604519, doi: 10.3389/fendo.2020.604519.
63. Vankelecom, H.; Roose, H. The Stem Cell Connection of Pituitary Tumors. Front Endocrinol (Lausanne) 2017, 8, 339, doi: 10.3389/fendo.2017.00339.
64. Mertens, F.; Gremeaux, L.; Chen, J.; Fu, Q.; Willems, C.; Roose, H.; Govaere, O.; Roskams, T.; Cristina, C.; Becu-Villalobos, D.; et al. Pituitary tumors contain a side population with tumor stem cell-associated characteristics. Endocr Relat Cancer 2015, 22, 481-504, doi: 10.1530/ERC-14-0546.
65. Chen, H. Z.; Tsai, S. Y.; Leone, G. Emerging roles of E2Fs in cancer: an exit from cell cycle control. Nat Rev Cancer 2009, 9, 785-797, doi: 10.1038/nrc2696.
66. Xu, Q.; Yuan, X.; Tunici, P.; Liu, G.; Fan, X.; Xu, M.; Hu, J.; Hwang, J. Y.; Farkas, D. L.; Black, K. L.; et al. Isolation of tumour stem-like cells from benign tumours. Br J Cancer 2009, 101, 303-311, doi: 10.1038/sj.bjc.6605142.
67. H. Cushing, Posterior Pituitary Activity from an Anatomical Standpoint, Am J Pathol, 9 (1933) 539-548 519.
68. H. Cushing, The basophil adenomas of the pituitary body and their clinical manifestations (pituitary basophilism). 1932, Obes Res, 2 (1994) 486-508.
69. N. Ironside, C. J. Chen, C. C. Lee, D. M. Trifiletti, M. L. Vance, J. P. Sheehan, Outcomes of Pituitary Radiation for Cushing's Disease, Endocrinol Metab Clin North Am, 47 (2018) 349-365.
70. D. L. Loriaux, Diagnosis and Differential Diagnosis of Cushing's Syndrome, N Engl J Med, 377 (2017) e3.
71. S. L. Asa, O. Mete, A. Perry, R. Y. Osamura, Overview of the 2022 WHO Classification of Pituitary Tumors, Endocr Pathol, 33 (2022) 6-26.
72. H. Nishioka, S. Yamada, Cushing's Disease, J Clin Med, 8 (2019).
73. R. A. Feelders, L. J. Hofland, Medical treatment of Cushing's disease, J Clin Endocrinol Metab, 98 (2013) 425-438.
74. P. Limumpornpetch, A. W. Morgan, A. Tiganescu, P. D. Baxter, V. Nyawira Nyaga, M. Pujades-Rodriguez, P. M. Stewart, The Effect of Endogenous Cushing Syndrome on All-cause and Cause-specific Mortality, J Clin Endocrinol Metab, 107 (2022) 2377-2388.
75. A. B. Atkinson, A. Kennedy, M. I. Wiggam, D. R. McCance, B. Sheridan, Long-term remission rates after pituitary surgery for Cushing's disease: the need for long-term surveillance, Clin Endocrinol (Oxf), 63 (2005) 549-559.
76. R. van der Pas, R. A. Feelders, F. Gatto, C. de Bruin, A. M. Pereira, P. M. van Koetsveld, D. M. Sprij-Mooij, A. M. Waaijers, F. Dogan, S. Schulz, J. M. Kros, S. W. Lamberts, L. J. Hofland, Preoperative normalization of cortisol levels in Cushing's disease after medical treatment: consequences for somatostatin and dopamine receptor subtype expression and in vitro response to somatostatin analogs and dopamine agonists, J Clin Endocrinol Metab, 98 (2013) E1880-1890.
77. D. Kondziolka, Cushing's disease and stereotactic radiosurgery, J Neurosurg, 119 (2013) 1484-1485; discussion 1485.
78. G. U. Mehta, J. P. Sheehan, M. L. Vance, Effect of stereotactic radiosurgery before bilateral adrenalectomy for Cushing's disease on the incidence of Nelson's syndrome, J Neurosurg, 119 (2013) 1493-1497.
79. M. Fleseriu, B. M. Biller, J. W. Findling, M. E. Molitch, D. E. Schteingart, C. Gross, S. S. Investigators, Mifepristone, a glucocorticoid receptor antagonist, produces clinical and metabolic benefits in patients with Cushing's syndrome, J Clin Endocrinol Metab, 97 (2012) 2039-2049.
80. J. Feiteiro, M. Mariana, I. Verde, E. Cairrao, Genomic and Nongenomic Effects of Mifepristone at the Cardiovascular Level: A Review, Reprod Sci, 24 (2017) 976-988.
81. H. Hunt, K. Donaldson, M. Strem, V. Zann, P. Leung, S. Sweet, A. Connor, D. Combs, J. Belanoff, Assessment of Safety, Tolerability, Pharmacokinetics, and Pharmacological Effect of Orally Administered CORT125134: An Adaptive, Double-Blind, Randomized, Placebo-Controlled Phase 1 Clinical Study, Clin Pharmacol Drug Dev, 7 (2018) 408-421.
82. H. J. Hunt, J. K. Belanoff, I. Walters, B. Gourdet, J. Thomas, N. Barton, J. Unitt, T. Phillips, D. Swift, E. Eaton, Identification of the Clinical Candidate (R)-(1-(4-Fluorophenyl)-6-((1-methyl-1H-pyrazol-4-yl) sulfonyl)-4,4a,5,6,7,8-hexah ydro-1H-pyrazolo[3,4-g] isoquinolin-4a-yl) (4-(trifluoromethyl) pyridin-2-yl) methano ne (CORT125134): A Selective Glucocorticoid Receptor (GR) Antagonist, J Med Chem, 60 (2017) 3405-3421.
83. M. Massimo Terzolo, Davide lacuaniello, MD, Anna Pia, M D, Priola Adriano, M D, Andreas Moraitis, M D, and Rosario Pivonello, M D, SUN-463 Tumor Shrinkage with Preoperative Relacorilant Therapy in Two Patients with Cushing Disease Due to Pituitary Macroadenomas, Journal of the Endocrine Society, (2019).
84. R. Pivonello, P. N. Munster, M. Terzolo, R. Ferrigno, C. Simeoli, S. Puglisi, U. Bali, A. G. Moraitis, Glucocorticoid Receptor Antagonism Upregulates Somatostatin Receptor Subtype 2 Expression in ACTH-Producing Neuroendocrine Tumors: New Insight Based on the Selective Glucocorticoid Receptor Modulator Relacorilant, Front Endocrinol (Lausanne), 12 (2021) 793262.
85. R. Pivonello, R. Ferrigno, M. C. De Martino, C. Simeoli, N. Di Paola, C. Pivonello, L. Barba, M. Negri, C. De Angelis, A. Colao, Medical Treatment of Cushing's Disease: An Overview of the Current and Recent Clinical Trials, Front Endocrinol (Lausanne), 11 (2020) 648.
86. F. Gatto, F. Barbieri, M. Arvigo, S. Thellung, J. Amaru, M. Albertelli, D. Ferone, T. Florio, Biological and Biochemical Basis of the Differential Efficacy of First and Second Generation Somatostatin Receptor Ligands in Neuroendocrine Neoplasms, Int J Mol Sci, 20 (2019).
87. R. A. Feelders, C. de Bruin, A. M. Pereira, J. A. Romijn, R. T. Netea-Maier, A. R. Hermus, P. M. Zelissen, R. van Heerebeek, F. H. de Jong, A. J. van der Lely, W. W. de Herder, L. J. Hofland, S. W. Lamberts, Pasireotide alone or with cabergoline and ketoconazole in Cushing's disease, N Engl J Med, 362 (2010) 1846-1848.
88. A. Lacroix, F. Gu, W. Gallardo, R. Pivonello, Y. Yu, P. Witek, M. Boscaro, R. Salvatori, M. Yamada, L. Tauchmanova, M. Roughton, S. Ravichandran, S. Petersenn, B. M. K. Biller, J. Newell-Price, G. S. G. Pasireotide, Efficacy and safety of once-monthly pasireotide in Cushing's disease: a 12 month clinical trial, Lancet Diabetes Endocrinol, 6 (2018) 17-26.
89. A. Colao, S. Petersenn, J. Newell-Price, J. W. Findling, F. Gu, M. Maldonado, U. Schoenherr, D. Mills, L. R. Salgado, B. M. Biller, B. S. G. Pasireotide, A 12-month phase 3 study of pasireotide in Cushing's disease, N Engl J Med, 366 (2012) 914-924.
90. K. H. Narsinh, F. Jia, R. C. Robbins, M. A. Kay, M. T. Longaker, J. C. Wu, Generation of adult human induced pluripotent stem cells using nonviral minicircle DNA vectors, Nat Protoc, 6 (2011) 78-88.
91. C. A. Stratakis, M. A. Tichomirowa, S. Boikos, M. F. Azevedo, M. Lodish, M. Martari, S. Verma, A. F. Daly, M. Raygada, M. F. Keil, J. Papademetriou, L. Drori-Herishanu, A. Horvath, K. M. Tsang, M. Nesterova, S. Franklin, J. F. Vanbellinghen, V. Bours, R. Salvatori, A. Beckers, The role of germline AIP, MEN1, PRKAR1A, CDKN1B and CDKN2C mutations in causing pituitary adenomas in a large cohort of children, adolescents, and patients with genetic syndromes, Clin Genet, 78 (2010) 457-463.
92. Z. Y. Ma, Z. J. Song, J. H. Chen, Y. F. Wang, S. Q. Li, L. F. Zhou, Y. Mao, Y. M. Li, R. G. Hu, Z. Y. Zhang, H. Y. Ye, M. Shen, X. F. Shou, Z. Q. Li, H. Peng, Q. Z. Wang, D. Z. Zhou, X. L. Qin, J. Ji, J. Zheng, H. Chen, Y. Wang, D. Y. Geng, W. J. Tang, C. W. Fu, Z. F. Shi, Y. C. Zhang, Z. Ye, W. Q. He, Q. L. Zhang, Q. S. Tang, R. Xie, J. W. Shen, Z. J. Wen, J. Zhou, T. Wang, S. Huang, H. J. Qiu, N. D. Qiao, Y. Zhang, L. Pan, W. M. Bao, Y. C. Liu, C. X. Huang, Y. Y. Shi, Y. Zhao, Recurrent gain-of-function USP8 mutations in Cushing's disease, Cell Res, 25 (2015) 306-317.
93. M. Reincke, S. Sbiera, A. Hayakawa, M. Theodoropoulou, A. Osswald, F. Beuschlein, T. Meitinger, E. Mizuno-Yamasaki, K. Kawaguchi, Y. Saeki, K. Tanaka, T. Wieland, E. Graf, W. Saeger, C. L. Ronchi, B. Allolio, M. Buchfelder, T. M. Strom, M. Fassnacht, M. Komada, Mutations in the deubiquitinase gene USP8 cause Cushing's disease, Nat Genet, 47 (2015) 31-38.
94. M. Reincke, M. Theodoropoulou, Genomics in Cushing's Disease: The Dawn of a New Era, J Clin Endocrinol Metab, 106 (2021) e2455-e2456.
95. J. Chakrabarti, R. Pandey, J. M. Churko, J. Eschbacher, S. Mallick, Y. Chen, B. Hermes, P. Mallick, B. N. Stansfield, K. W. Pond, C. A. Thorne, K. C. J. Yuen, A. S. Little, Y. Zavros, Development of Human Pituitary Neuroendocrine Tumor Organoids to Facilitate Effective Targeted Treatments of Cushing's Disease, Cells, 11 (2022).
96. N. G. Steele, J. Chakrabarti, J. Wang, J. Biesiada, L. Holokai, J. Chang, L. M. Nowacki, J. Hawkins, M. Mahe, N. Sundaram, N. Shroyer, M. Medvedovic, M. Helmrath, S. Ahmad, Y. Zavros, An Organoid-Based Preclinical Model of Human Gastric Cancer, Cell Mol Gastroenterol Hepatol, 7 (2019) 161-184.
97. R. Feng, E. Aihara, S. Kenny, L. Yang, J. Li, A. Varro, M. H. Montrose, N. F. Shroyer, T. C. Wang, R. A. Shivdasani, Y. Zavros, Indian Hedgehog mediates gastrin-induced proliferation in stomach of adult mice, Gastroenterology, 147 (2014) 655-666 e659.
98. E. C. Filippi-Chiela, M. M. Oliveira, B. Jurkovski, S. M. Callegari-Jacques, V. D. da Silva, G. Lenz, Nuclear morphometric analysis (NMA): screening of senescence, apoptosis and nuclear irregularities, PLOS One, 7 (2012) e42522.
99. K. J. Livak, T. D. Schmittgen, Analysis of relative gene expression data using real-time quantitative PCR and the 2 (−Delta Delta C (T)) Method, Methods, 25 (2001) 402-408.
100 J. Chen, X. Jian, S. Deng, Z. Ma, X. Shou, Y. Shen, Q. Zhang, Z. Song, Z. Li, H. Peng, C. Peng, M. Chen, C. Luo, D. Zhao, Z. Ye, M. Shen, Y. Zhang, J. Zhou, A. Fahira, Y. Wang, S. Li, Z. Zhang, H. Ye, Y. Li, J. Shen, H. Chen, F. Tang, Z. Yao, Z. Shi, C. Chen, L. Xie, Y. Wang, C. Fu, Y. Mao, L. Zhou, D. Gao, H. Yan, Y. Zhao, C. Huang, Y. Shi, Identification of recurrent USP48 and BRAF mutations in Cushing's disease, Nat Commun, 9 (2018) 3171.
101. B. Zimmer, J. Piao, K. Ramnarine, M. J. Tomishima, V. Tabar, L. Studer, Derivation of Diverse Hormone-Releasing Pituitary Cells from Human Pluripotent Stem Cells, Stem Cell Reports, 6 (2016) 858-872.
102. X. Zhu, A. S. Gleiberman, M. G. Rosenfeld, Molecular physiology of pituitary development: signaling and transcriptional networks, Physiol Rev, 87 (2007) 933-963.
103. C. Ballmann, A. Thiel, H. E. Korah, A. C. Reis, W. Saeger, S. Stepanow, K. Kohrer, G. Reifenberger, C. B. Knobbe-Thomsen, U. J. Knappe, U. I. Scholl, USP8 Mutations in Pituitary Cushing Adenomas-Targeted Analysis by Next-Generation Sequencing, J Endocr Soc, 2 (2018) 266-278.
104 T. Sahakitrungruang, C. Srichomthong, S. Pornkunwilai, J. Amornfa, S. Shuangshoti, S. Kulawonganunchai, K. Suphapeetiporn, V. Shotelersuk, Germline and somatic DICER1 mutations in a pituitary blastoma causing infantile-onset Cushing's disease, J Clin Endocrinol Metab, 99 (2014) E1487-1492.
105. F. Cetani, E. Pardi, L. Cianferotti, E. Vignali, A. Picone, P. Miccoli, A. Pinchera, C. Marcocci, A new mutation of the MEN1 gene in an italian kindred with multiple endocrine neoplasia type 1, Eur J Endocrinol, 140 (1999) 429-433.
106. F. Marini, F. Giusti, C. Fossi, F. Cioppi, L. Cianferotti, L. Masi, F. Boaretto, S. Zovato, F. Cetani, A. Colao, M. V. Davi, A. Faggiano, G. Fanciulli, P. Ferolla, D. Ferone, P. Loli, F. Mantero, C. Marcocci, G. Opocher, P. Beck-Peccoz, L. Persani, A. Scillitani, F. Guizzardi, A. Spada, P. Tomassetti, F. Tonelli, M. L. Brandi, Multiple endocrine neoplasia type 1: analysis of germline MEN1 mutations in the Italian multicenter MEN1 patient database, Endocrine, 62 (2018) 215-233.
107 L. Cazabat, J. Bouligand, P. Chanson, AIP mutation in pituitary adenomas, N Engl J Med, 364 (2011) 1973-1974; author reply 1974-1975.
108. L. Cazabat, J. Bouligand, S. Salenave, M. Bernier, S. Gaillard, F. Parker, J. Young, A. Guiochon-Mantel, P. Chanson, Germline AIP mutations in apparently sporadic pituitary adenomas: prevalence in a prospective single-center cohort of 443 patients, J Clin Endocrinol Metab, 97 (2012) E663-670.
109. S. Sbiera, L. G. Perez-Rivas, L. Taranets, I. Weigand, J. Flitsch, E. Graf, C. M. Monoranu, W. Saeger, C. Hagel, J. Honegger, G. Assie, A. R. Hermus, G. K. Stalla, S. Herterich, C. L. Ronchi, T. Deutschbein, M. Reincke, T. M. Strom, N. Popov, M. Theodoropoulou, M. Fassnacht, Driver mutations in USP8 wild-type Cushing's disease, Neuro Oncol, 21 (2019) 1273-1283.
110. A. Zhou, K. Lin, S. Zhang, L. Ma, J. Xue, S. A. Morris, K. D. Aldape, S. Huang, Gli1-induced deubiquitinase USP48 aids glioblastoma tumorigenesis by stabilizing Gli1, EMBO Rep, 18 (2017) 1318-1330.
111. G. Vila, M. Papazoglou, J. Stalla, M. Theodoropoulou, G. K. Stalla, F. Holsboer, M. Paez-Pereda, Sonic hedgehog regulates CRH signal transduction in the adult pituitary, FASEB J, 19 (2005) 281-283.
112. G. Vila, M. Theodoropoulou, J. Stalla, J. C. Tonn, M. Losa, U. Renner, G. K. Stalla, M. Paez-Pereda, Expression and function of sonic hedgehog pathway components in pituitary adenomas: evidence for a direct role in hormone secretion and cell proliferation, J Clin Endocrinol Metab, 90 (2005) 6687-6694.
113. R. Xia, H. Jia, J. Fan, Y. Liu, J. Jia, USP8 promotes smoothened signaling by preventing its ubiquitination and changing its subcellular localization, PLOS Biol, 10 (2012) e1001238.
114. J. Pyczek, R. Buslei, D. Schult, A. Holsken, M. Buchfelder, I. Hess, H. Hahn, A. Uhmann, Hedgehog signaling activation induces stem cell proliferation and hormone release in the adult pituitary gland, Sci Rep, 6 (2016) 24928.
115. D. Brennan, X. Chen, L. Cheng, M. Mahoney, N. A. Riobo, Noncanonical Hedgehog signaling, Vitam Horm, 88 (2012) 55-72.
116. R. Teperino, F. Aberger, H. Esterbauer, N. Riobo, J. A. Pospisilik, Canonical and non-canonical Hedgehog signalling and the control of metabolism, Semin Cell Dev Biol, 33 (2014) 81-92.
117. D. Bongiovanni, V. Tosello, V. Saccomani, S. Dalla Santa, A. Amadori, P. Zanovello, E. Piovan, Crosstalk between Hedgehog pathway and the glucocorticoid receptor pathway as a basis for combination therapy in T-cell acute lymphoblastic leukemia, Oncogene, 39 (2020) 6544-6555.
118. C. Xiao, R. Feng, A. Engevik, J. Martin, J. Tritschler, M. Schumacher, R. Koncar, J. Roland, K. 118. Nam, J. Goldenring, Y. Zavros, Sonic Hedgehog contributes to gastric mucosal restitution after injury, Lab Invest, 93 (2013) 96-111. 119.
119. C. Xiao, S. A. Ogle, M. A. Schumacher, M. A. Orr-Asman, M. L. Miller, N. Lertkowit, A. Varro, F. Hollande, Y. Zavros, Loss of Parietal Cell Expression of Sonic Hedgehog Induces Hypergastrinemia and Hyperproliferation of Surface Mucous Cells., Gastroenterology, 138 (2010) 550-561.
120. M. Treier, S. O'Connell, A. Gleiberman, J. Price, D. P. Szeto, R. Burgess, P. T. Chuang, A. P. McMahon, M. G. Rosenfeld, Hedgehog signaling is required for pituitary gland development, Development, 128 (2001) 377-386.
121 J. Tang, L. Chen, Z. Wang, G. Huang, X. Hu, SOX2 mediates crosstalk between Sonic Hedgehog and the Wnt/beta-catenin signaling pathway to promote proliferation of pituitary adenoma cells, Oncol Lett, 18 (2019) 81-86.
122 D. S. Botermann, N. Brandes, A. Frommhold, I. Hess, A. Wolff, A. Zibat, H. Hahn, R. Buslei, A. Uhmann, Hedgehog signaling in endocrine and folliculo-stellate cells of the adult pituitary, J Endocrinol, 248 (2021) 303-316.
123. M. Fleseriu, J. W. Findling, C. A. Koch, S. M. Schlaffer, M. Buchfelder, C. Gross, Changes in plasma ACTH levels and corticotroph tumor size in patients with Cushing's disease during long-term treatment with the glucocorticoid receptor antagonist mifepristone, J Clin Endocrinol Metab, 99 (2014) 3718-3727.
124. M. Grant, H. Alturaihi, P. Jaquet, B. Collier, U. Kumar, Cell growth inhibition and functioning of human somatostatin receptor type 2 are modulated by receptor heterodimerization, Mol Endocrinol, 22 (2008) 2278-2292.
125. M. Cakir, D. Dworakowska, A. Grossman, Somatostatin receptor biology in neuroendocrine and pituitary tumours: part 1—molecular pathways, J Cell Mol Med, 14 (2010) 2570-2584.
126. N. Sharif, L. Gendron, J. Wowchuk, P. Sarret, J. Mazella, A. Beaudet, T. Stroh, Coexpression of somatostatin receptor subtype 5 affects internalization and trafficking of somatostatin receptor subtype 2, Endocrinology, 148 (2007) 2095-2105.
127. N. Ahmad, R. Kumar, Steroid hormone receptors in cancer development: a target for cancer therapeutics, Cancer Lett, 300 (2011) 1-9.
128. P. N. Munster, A. E. Greenstein, G. F. Fleming, E. Borazanci, M. R. Sharma, J. M. Custodio, I. C. Tudor, H. I. Pashova, S. Peacock Shepherd, A. Grauer, J. C. Sachdev, Overcoming Taxane Resistance: Preclinical and Phase 1 Studies of Relacorilant, a Selective Glucocorticoid Receptor Antagonist, with Nab-Paclitaxel in Solid Tumors, Clin Cancer Res, (2022).
129. A. E. Greenstein, H. J. Hunt, Glucocorticoid receptor antagonism promotes apoptosis in solid tumor cells, Oncotarget, 12 (2021) 1243-1255.
130. A. L. Gruver-Yates, J. A. Cidlowski, Tissue-specific actions of glucocorticoids on apoptosis: a double-edged sword, Cells, 2 (2013) 202-223.
131 L. Holokai, J. Chakrabarti, J. Lundy, D. Croagh, P. Adhikary, S. S. Richards, C. Woodson, N. Steele, R. Kuester, A. Scott, M. Khreiss, T. Frankel, J. Merchant, B. J. Jenkins, J. Wang, R. T. Shroff, S. A. Ahmad, Y. Zavros, Murine- and Human-Derived Autologous Organoid/Immune Cell Co-Cultures as Pre-Clinical Models of Pancreatic Ductal Adenocarcinoma, Cancers (Basel), 12 (2020).
132. S. F. Boj, C. I. Hwang, L. A. Baker, Chio, II, D. D. Engle, V. Corbo, M. Jager, M. Ponz-Sarvise, H. Tiriac, M. S. Spector, A. Gracanin, T. Oni, K. H. Yu, R. van Boxtel, M. Huch, K. D. Rivera, J. P. Wilson, M. E. Feigin, D. Ohlund, A. Handly-Santana, C. M. Ardito-Abraham, M. Ludwig, E. Elyada, B. Alagesan, G. Biffi, G. N. Yordanov, B. Delcuze, B. Creighton, K. Wright, Y. Park, F. H. Morsink, I. Q. Molenaar, I. H. Borel Rinkes, E. Cuppen, Y. Hao, Y. Jin, I. J. Nijman, C. lacobuzio-Donahue, S. D. Leach, D. J. Pappin, M. Hammell, D. S. Klimstra, O. Basturk, R. H. Hruban, G. J. Offerhaus, R. G. Vries, H. Clevers, D. A. Tuveson, Organoid models of human and mouse ductal pancreatic cancer, Cell, 160 (2015) 324-338.
133. H. Tiriac, P. Belleau, D. D. Engle, D. Plenker, A. Deschenes, T. D. D. Somerville, F. E. M. Froeling, R. A. Burkhart, R. E. Denroche, G. H. Jang, K. Miyabayashi, C. M. Young, H. Patel, M. Ma, J. F. LaComb, R. L. D. Palmaira, A. A. Javed, J. C. Huynh, M. Johnson, K. Arora, N. Robine, M. Shah, R. Sanghvi, A. B. Goetz, C. Y. Lowder, L. Martello, E. Driehuis, N. LeComte, G. Askan, C. A. lacobuzio-Donahue, H. Clevers, L. D. Wood, R. H. Hruban, E. Thompson, A. J. Aguirre, B. M. Wolpin, A. Sasson, J. Kim, M. Wu, J. C. Bucobo, P. Allen, D. V. Sejpal, W. Nealon, J. D. Sullivan, J. M. Winter, P. A. Gimotty, J. L. Grem, D. J. DiMaio, J. M. Buscaglia, P. M. Grandgenett, J. R. Brody, M. A. Hollingsworth, G. M. O'Kane, F. Notta, E. Kim, J. M. Crawford, C. Devoe, A. Ocean, C. L. Wolfgang, K. H. Yu, E. Li, C. R. Vakoc, B. Hubert, S. E. Fischer, J. M. Wilson, R. Moffitt, J. Knox, A. Krasnitz, S. Gallinger, D. A. Tuveson, Organoid Profiling Identifies Common
134 Responders to Chemotherapy in Pancreatic Cancer, Cancer Discov, 8 (2018) 1112-1129. D. C. Danila, X. Zhang, Y. Zhou, G. R. Dickersin, J. A. Fletcher, E. T. Hedley-Whyte, M. K. Selig, S. R. Johnson, A. Klibanski, A human pituitary tumor-derived folliculostellate cell line, J Clin Endocrinol Metab, 85 (2000) 1180-1187.
135. T. Bjoro, P. A. Torjesen, B. C. Ostberg, O. Sand, J. G. Iversen, K. M. Gautvik, E. Haug, Bombesin stimulates prolactin secretion from cultured rat pituitary tumour cells (GH4C1) via activation of phospholipase C, Regul Pept, 19 (1987) 169-182. 136.
136. T. Bjoro, O. Sand, B. C. Ostberg, J. O. Gordeladze, P. Torjesen, K. M. Gautvik, E. Haug, The mechanisms by which vasoactive intestinal peptide (VIP) and thyrotropin releasing hormone (TRH) stimulate prolactin release from pituitary cells, Biosci Rep, 10 (1990) 189-199.
137. H. Ikeda, T. Mitsuhashi, K. Kubota, N. Kuzuya, H. Uchimura, Epidermal growth factor stimulates growth hormone secretion from superfused rat adenohypophyseal fragments, Endocrinology, 115 (1984) 556-558.
138. N. Baek, O. W. Seo, M. Kim, J. Hulme, S. S. An, Monitoring the effects of doxorubicin on 3D-spheroid tumor cells in real-time, Onco Targets Ther, 9 (2016) 7207-7218.
139. B. Cox, E. Laporte, A. Vennekens, H. Kobayashi, C. Nys, I. Van Zundert, I. H. Uji, A. Vercauteren Drubbel, B. Beck, H. Roose, M. Boretto, H. Vankelecom, Organoids from pituitary as a novel research model toward pituitary stem cell exploration, J Endocrinol, 240 (2019) 287-308.
140. A. Vennekens, E. Laporte, F. Hermans, B. Cox, E. Modave, A. Janiszewski, C. Nys, H. Kobayashi, B. Malengier-Devlies, J. Chappell, P. Matthys, M. I. Garcia, V. Pasque, D. Lambrechts, H. Vankelecom, Interleukin-6 is an activator of pituitary stem cells upon local damage, a competence quenched in the aging gland, Proc Natl Acad Sci USA, 118 (2021).
141 E. Laporte, C. Nys, H. Vankelecom, Development of Organoids from Mouse Pituitary as In Vitro Model to Explore Pituitary Stem Cell Biology, J Vis Exp, (2022).
142. C. Nys, Y. L. Lee, H. Roose, F. Mertens, E. De Pauw, H. Kobayashi, R. Sciot, M. Bex, G. Versyck, S. De Vleeschouwer, J. Van Loon, E. Laporte, H. Vankelecom, Exploring stem cell biology in pituitary tumors and derived organoids, Endocr Relat Cancer, 29 (2022) 427-450.
143. M. M. Mahe, E. Aihara, M. A. Schumacher, Y. Zavros, M. H. Montrose, M. A. Helmrath, T. Sato, N. F. Shroyer, Establishment of Gastrointestinal Epithelial Organoids, Current Protocols in Mouse Biology, 3 (2013) 217-240.
144. M. A. Schumacher, E. Aihara, R. Feng, A. Engevik, N. F. Shroyer, K. M. Ottemann, R. T. Worrell, M. H. Montrose, R. A. Shivdasani, Y. Zavros, The use of murine-derived fundic organoids in studies of gastric physiology, J Physiol, 593 (2015) 1809-1827.
145. A. Shintani, M. Higuchi, Isolation of PRRX1-positive adult pituitary stem/progenitor cells from the marginal cell layer of the mouse anterior lobe, Stem Cell Res, 52 (2021) 102223.
146. R. Wurth, F. Barbieri, A. Pattarozzi, G. Gaudenzi, F. Gatto, P. Fiaschi, J. L. Ravetti, G. Zona, A. Daga, L. Persani, D. Ferone, G. Vitale, T. Florio, Phenotypical and Pharmacological Characterization of Stem-Like Cells in Human Pituitary Adenomas, Mol Neurobiol, 54 (2017) 4879-4895.
147. R. Wurth, S. Thellung, A. Corsaro, F. Barbieri, T. Florio, Experimental Evidence and Clinical Implications of Pituitary Adenoma Stem Cells, Front Endocrinol (Lausanne), 11 (2020) 54.
148. T. Kasai, H. Suga, M. Sakakibara, C. Ozone, R. Matsumoto, M. Kano, K. Mitsumoto, K. Ogawa, Y. Kodani, H. Nagasaki, N. Inoshita, M. Sugiyama, T. Onoue, T. Tsunekawa, Y. Ito, H. Takagi, D. Hagiwara, S. Iwama, M. Goto, R. Banno, J. Takahashi, H. Arima, Hypothalamic Contribution to Pituitary Functions Is Recapitulated In Vitro Using 3D-Cultured Human iPS Cells, Cell Rep, 30 (2020) 18-24 e15.
149. G. Mantovani, E. Giardino, D. Treppiedi, R. Catalano, F. Mangili, A. Spada, M. Arosio, E. Peverelli, Stem Cells in Pituitary Tumors: Experimental Evidence Supporting Their Existence and Their Role in Tumor Clinical Behavior, Front Endocrinol (Lausanne), 10 (2019) 745.
150. Y. Zhou, R. R. A. Wilson, A. Udaiyar, J. Mclemore, H. Sadri-Ardekani, T. Criswell, Pituitary lineage differentiation from human-induced pluripotent stem cells in 2D and 3D cultures, Stem Cells Dev, (2022).
151. D. Zhang, W. Hugo, P. Redublo, H. Miao, M. Bergsneider, M. B. Wang, W. Kim, W. H. Yong, A. P. Heaney, A human ACTH-secreting corticotroph tumoroid model: Novel Human ACTH-Secreting Tumor Cell in vitro Model, EBioMedicine, 66 (2021) 103294.

We claim:

1. An organoid derived from a pituitary neuroendocrine tumor (PitNET) of a human subject, wherein the organoid comprises a plurality of cells that is well differentiated, and wherein the organoid possesses structure and function similar to those of the PitNET.

2. The organoid of claim 1, wherein the PitNET is selected from the group consisting of a corticotroph adenoma, a lactotroph adenoma, a gonadotroph adenoma, and a somatotroph adenoma.

3. The organoid of claim 1, wherein the PitNET is a corticotroph adenoma.

4. The organoid of claim 3, wherein the PitNET secrets adrenocorticotropic hormone (ACTH).

5. The organoid of claim 1, wherein at least some cells of the organoid retain genetic alteration(s) of the human subject's primary tissue.

6. The organoid of claim 1, wherein at least some cells of the organoid carry an M415V and/or an M415I mutation in the USP48 gene.

7. The organoid of claim 1, wherein the PitNET is from a subject that carries a mutant allele selected from the group consisting of CDH23 and MEN1.

8. A method of treating Cushing's Disease (CD) caused by a pituitary neuroendocrine tumor (PitNET) in a subject, comprising
a) extracting a plurality of cells from the PitNET of the subject,
b) culturing the plurality of cells to form an organoid,
c) contacting the organoid with a compound selected from a plurality of compounds,
d) determining if the compound increases apoptosis or reduces hormonal secretion of the organoid after being contacted with the compound, and
e) selecting the compound that increases apoptosis or reduces hormonal secretion of the organoid as a candidate compound,
f) administering the candidate compound to the subject.

9. The method of claim 8, wherein steps (c) and (d) are repeated until all compounds from the plurality of compounds have gone through steps (c) and (d).

10. The method of claim 8, wherein the candidate compound binds to human glucocorticoid receptor (GR) but does not bind to other non-GR hormone receptors.

11. The method of claim 8, wherein the candidate compound is administered together with a pharmaceutically acceptable carrier.

12. An organoid comprising a plurality of cells derived from a human induced pluripotent stem cells (iPSC) lines, wherein somatic mutations M415V and/or M415I in the gene USP48 have been introduced into certain cells of the organoid.

13. The organoid of claim 12, wherein the somatic mutations M415V and/or M415I are introduced by CRISPR.

14. A method of screening for compounds that have therapeutic effects for Cushing's Disease (CD) in human, comprising
a) contacting the organoid of claim 1 with one or more compounds, and
b) assessing apoptosis or hormonal secretion profile of the organoid after the contacting step (a),
c) identifying a candidate compound that induces apoptosis or reduces ACTH secretion.

15. A method of screening for compounds that have therapeutic effects for Cushing's Disease (CD) in human, comprising
a) contacting the organoid of claim 12 with one or more compounds, and
b) assessing apoptosis or hormonal secretion profile of the organoid after the contacting step (a),
c) identifying a candidate compound that induces apoptosis or reduces ACTH secretion.
